(19) United States
(12) Patent Application Publication
(10) Pub. No.: US 2010/0273259 A1
(43) Pub. Date: Oct. 28, 2010

(54) SUBSTRATES AND METHODS FOR CULTURING STEM CELLS

(75) Inventors: Krishanu Saha, Cambridge, MA (US); Ying Mei, Cambridge, MA (US); Said R. Bogatyrev, Cambridge, MA (US); Daniel G. Anderson, Sudbury, MA (US); Rudolf Jaenisch, Brookline, MA (US); Robert S. Langer, Newton, MA (US); Morgan Alexander, Nottingham (GB); Martyn Davies, Nottingham (GB); Jing Yang, Nottingham (GB); Christian J. Kastrup, Cambridge, MA (US); Andrew Urquhart, Glasgow (GB)

Correspondence Address: SUTHERLAND ASBILL & BRENNAN LLP, 999 PEACHTREE STREET, N.E., ATLANTA, GA, 30309, US

(73) Assignee: MASSACHUSETTS INSTITUTE OF TECHNOLOGY, Cambridge, MA (US)

(21) Appl. No.: 12/765,472

(22) Filed: Apr. 22, 2010

Related U.S. Application Data
(60) Provisional application No. 61/171,715, filed on Apr. 22, 2009.

(51) Int. Cl. C12N 5/0797

(52) U.S. Cl. 435/377; 435/395; 435/396

(57) ABSTRACT

The present disclosure provides a device and a cell culture system comprising a substrate that generates significant chemical ion signatures adapted for culturing stem cells. This disclosure further provides unique surface properties, such as surface wettability, along with defined polymer microspot environments in an array, for effectively supporting the propagation and differentiation of human pluripotent stem cells in vitro. Methods of culturing, maintenance, differentiating stem cells as well as reprogramming somatic cells into stem cells using the device and the cell culture system with the suitable substrates, along with suitable culture media, are also provided.

### CROSS REFERENCE OF RELATED APPLICATIONS

This application claims priority to and the benefit of U.S. Provisional Patent Application No. 61/171,715 filed Apr. 22, 2009, which is hereby incorporated by reference in its entirety.

### GOVERNMENT SUPPORT STATEMENT

This invention was made with government support under DE016516-03 and R37CA084198 awarded by National Institutions of Health. The government has certain rights in the invention.

### BACKGROUND OF THE INVENTION

1. Field of the Invention

This disclosure relates generally to a device or a culture system containing biomaterials as substrates, and method of use thereof, for culturing mammalian multipotent and pluripotent stem cells, particularly, for supporting the expansion, somatic cell reprogramming, gene targeting, and differentiation of human multipotent and pluripotent stem cells (hPSCs).

2. Background Art

Both human embryonic stem (hES) cells and recently “reprogrammed” induced pluripotent stem (hiPS) cells can self-renew indefinitely in culture¹⁻⁴, however current methods to clonally grow them are inefficient and poorly-defined for genetic manipulation and therapeutic purposes⁵⁻⁷. Proof-of-principle experiments with mouse pluripotent stem cells⁸⁻¹¹ indicate that human pluripotent stem cells (both hES and hiPS cells) may hold great promise for regenerative medicine^{12,13} and human disease modeling¹⁴⁻¹⁶.

However the capabilities with human cells are not the same as those with mouse cells: mouse pluripotent stem cells can more easily be genetically engineered and their derivatives can be readily transplanted, while hES and hiPS cells cannot be genetically modified efficiently^{7,17,18}, and transplantation of their derivatives into human hosts is accompanied by safety issues^{13,19}. Improved human cell culture systems have the potential to address both issues, as existing methods to grow human pluripotent stem cells are both poorly suited for genetic engineering experiments and introduce animal components, increasing the risks of immune rejection.

Current methods include growing hES and hiPS cells on a “feeder” cell layer of mitotically-inactivated mouse embryo fibroblasts (mEFs)¹⁻⁴, and on “feeder-free” (i.e., cell-free) culture systems, composed of a variety of extracellular matrix (ECM)/serum proteins coated onto tissue culture dishes²⁰⁻²⁸ or synthetic materials^{29,30} like hyaluronic acid hydrogels. These have been reported to promote hES cell self-renewal when seeded at a suitably high cell density (e.g., ˜10⁶ cells/ml for the hydrogel)^{23,29,30}, and have not been demonstrated to efficiently promote clonal growth of single hES cells (efficiencies typically <10%). However, gene targeting in pluripotent stem cells necessitates clonal outgrowth of single cells to detect rare targeting events (1 in 10⁵-10⁶ cells) and requires selective growth of a correctly gene-targeted cell within a population of >10⁵ cells.^{7,17,18} Such clonal growth is highly efficient in cell culture systems used for mouse pluripotent stem cells in contrast to human pluripotent cells, likely impeding efficient gene manipulation in the latter. Further, current human culture methods utilize either animal products or undefined components, which make it problematic for the potential transplantation applications^{5,6,31,32}.

Therefore, human pluripotent stem cells (hPSCs), including human embryonic stem cells (hESCs) and human induced pluripotent stem cells (hiPSCs) hold the great promise for regenerative medicine. These cells can replicate indefinitely in culture and they can differentiate into almost any cell type in the human body. A significant challenge to use hPSCs in therapy is that they are technically difficult to culture, showing problematic properties such as slow growth and vulnerability to apoptosis upon complete dissociation. They tend to undergo massive cell death after complete dissociation, and this makes it challenging to genetically manipulate these cells and direct their differentiation. In addition, hPSCs are traditionally cultured on a layer of mitotically inactivated mouse embryo fibroblasts (MEFs), and the production of MEFs is highly laborious and limits the large-scale production of hPSCs. Furthermore, the animal origin of MEFs brings in the risks of animal pathogens and immunogenic animal proteins.

Although a variety of feeder free culture systems based on extracellular matrix (ECM) proteins including fibronectin, laminin and vitronectin were reported to maintain the long term culture of hESCs, recent reports indicate that the performance of these feeder free culture systems is inferior to the undefined, xenogenic matrigel. In addition, almost all the feeder free cell culture systems failed to maintain a long term culture for karyotypic normal hESCs. There is a need, therefore, to develop improved stem cell culture systems.

### SUMMARY OF THE INVENTION

In one aspect, the present disclosure provides devices and methods to overcome, or at least alleviate, one or more of the difficulties or deficiencies associated with the prior art. In that regard, this disclosure provides a device, and methods of use thereof, comprising substrates having surface ion signatures and optimal surface energies that support culturing, expansion, differentiation, gene targeting of stem cells, as well as reprogramming somatic cells to stem cells. The devices and methods can preserve normal karyotypes, and maintain differentiation capacity after prolonged cell culture. The present devices and methods provide chemically-defined, xeno-free, feeder-free substrates to support efficient clonal growth of stem cells, such as human pluripotent stem cells.

Therefore, this disclosure contemplates a device comprising a substrate adapted for culturing human multipotent and pluripotent stem cells and characterized by a secondary ion mass spectrometry (SIMS) ion signature corresponding to a predetermined ion signature correlated with a desired behavior in the stem cells, and wherein the substrate is untreated, or alternatively treated to generate the predetermined SIMS ion signature. In certain embodiments, the substrate comprises a polymer or an array of polymer domains distributed on a support. Generally, throughout this disclosure the term “polymer” is used generally to describe homopolymers, copolymers, and polymers of any number of monomers.

In certain embodiments, the substrate polymer comprises a polymer which is characterized by a secondary ion mass spectrometry (SIMS) ion signature comprising at least one of the three most intense ion peaks selected from a hydrocarbon ion having no tertiary carbon atoms, a cyclic hydrocarbon ion, or an oxygen-containing ion derived from an ester. In certain embodiments, the substrate comprises an acrylate-based polymer having a SIMS ion signature comprising at least one of the three most intense ion peaks selected from a C₁₋₄ hydrocarbon ion having no tertiary carbon atoms, a cyclic hydrocarbon ion, an oxygen-containing ion derived from an ester, O⁻, and OH⁻.

In some embodiments, the SIMS ion signature comprises at least one of the three most intense ion peaks selected from O⁻, C₂H⁻, OH⁻, CHO₂⁻, C₂H₃⁻, C₃H₅⁺, C₄H⁻, C₁₀H₁₁O⁻, CH⁻, C₃H₃⁻, C₃H₇⁺, C₂H₅O⁺, and C₂H₃O⁺. In some embodiments, the SIMS ion signature comprises a base peak selected from O⁻, C₂H⁻, OH⁻, CHO₂⁻, C₂H₃⁻, C₃H₅⁺, C₄H⁻, C₁₀H₁₁O⁻, CH⁻, C₃H₃⁻, C₃H₇⁺, C₂H₅O⁺, and C₂H₃O⁺, and at least one of the two subsequent ions according to peak intensity selected from O⁻, C₂H⁻, OH⁻, CHO₂⁻, C₂H₃⁻, C₃H₅⁺, C₄H⁻, C₁₀H₁₁O⁻, CH⁻, C₃H₃⁻, C₃H₇⁺, C₂H₅O⁺, and C₂H₃O⁺. In certain embodiments, the acrylate-based polymer or copolymer described in this disclosure has a SIMS ion signature comprising the three most intense ion peaks selected from an ion other than CN⁻, C₂H₇O⁺, C₄H₉⁺, C₂H₆N⁺, C₃H₃O₂⁻, C₃H₈N⁺, C₅H₉⁺, C₅H₁₁⁺, CNO⁻, and C₃H₇O⁺. In some embodiments, the acrylate-based polymer or copolymer of the present disclosure has a SIMS ion signature comprising the base peak selected from an ion other than CN⁻, C₂H₇O⁺, C₄H₉⁺, C₂H₆N⁺, C₃H₃O₂⁻, C₃H₈N⁺, C₅H₉⁺, C₅H₁₁⁺, CNO⁻, and C₃H₇O⁺.

In various embodiments, the polymer can be a styrene-based polymer having a SIMS ion signature comprising at least one of the three most intense ion peaks selected from a C₂₋₆ hydrocarbon ion having no tertiary carbon atoms, a cyclic hydrocarbon ion, or an oxygen-containing ion derived from an ester. In certain embodiments, for example in a styrene-based polymer substrate, the SIMS ion signature can comprise at least one of the three most intense ion peaks characterized by a carbon-to-hydrogen atomic ratio of less than 1. In some embodiments, the SIMS ion signature can comprise at least two of the three most intense ion peaks characterized by a carbon-to-hydrogen atomic ratio of less than 1.

In certain embodiments, the SIMS ion signature comprises at least one of the three most intense ion peaks selected from C₂H₄O⁺, C₆H₉O⁺, C₃H₃O⁺, C₂H₃⁺, C₆H₁₁⁺, C₂H₅⁺, C₂H₃O⁺, C₅H₇O⁺, and C₃H₅⁺. In some embodiments, the SIMS ion signature comprises a base peak selected from C₂H₄O⁺, C₆H₉O⁺, C₃H₃O⁺, C₂H₃⁺, C₂F⁻, C₆H₁₁⁺, C₂H₅⁺, C₂H₃O⁺, C₅H₇O⁺, and C₃H₅⁺, and at least one of the two subsequent ions according to peak intensity selected from C₂H₄O⁺, C₆H₉O⁺, C₃H₃O⁺, C₂H₃⁺, C₂F⁻, C₆H₁₁⁺, C₂H₅⁺, C₂H₃O⁺, C₅H₇O⁺, and C₃H₅⁺.

In certain embodiments, the styrene-based polymer of the present disclosure has a SIMS ion signature comprising at least one of the three most intense ion peaks selected from an ion other than C₇H₇⁺, CHO₂⁻, C₉H₉⁺, O⁻, C₇H₅O⁺, C₉H₇⁺, C₆H₅⁺, C₂H⁻, C₈H₇⁺, and C₇H₇O⁺. In some embodiments, the styrene-based polymer of this disclosure has a SIMS ion signature that comprises the base peak selected from an ion other than C₇H₇⁺, CHO₂⁻, C₉H₉⁺, O⁻, C₇H₅O⁺, C₉H₇⁺, C₆H₅⁺, C₂H⁻, C₈H₇⁺, and C₇H₇O⁺.

Besides molecular surface chemistry ion signatures of the substrates, the present disclosure further provides that other properties of the substrates including surface wettability and/or optimal surface energy, e.g., water contact angle, and the confined environments created by the micrometer scale spots, in particular their periphery, are useful aspects to support culturing stem cells. This disclosure thus contemplates that the unique combination defined by the surface properties and confined environments can effectively support culturing stem cells.

In certain embodiments, the device of the present disclosure comprises a substrate array, which comprises at least 10 polymer domains distributed on a support, and each domain has a moderate wettability with a water contact angle (WCA) of about 45° to 90° C. In certain embodiments, each polymer domain has a moderate wettability with a water contact angle (WCA) of about 55° to 80° C.

In some embodiments, the array of polymer domains comprises a repeating microenvironment array adapted for culturing stem cells, including human embryonic stem cells and induced pluripotent stem cells. Each microenvironment comprises the peripheral aspect of each polymer microspots having a major axis in a range of about 1 μm to 1000 μm. In certain embodiments, the major axis of each polymer microspots is in a range of about 10 μm-500 μm; alternatively in a range of about 100 μm-450 μm; or alternatively, in a range of about 200 μm-400 μm.

Therefore, this disclosure provides that substrates to support, maintain, and promote stem cell growth and differentiation can be generated from monomers, and that a plurality of such suitable substrates can be used to fabricate arrays. Results were validated from primary screening, which further confirmed their capacity to maintain pluripotency of stem cells, preserve normal karyotype, and maintain differentiation capacity after prolonged cell culture. Moreover, the efficacy of substrate microspots to support single cell growth of stem cells were found to be similar to MEFs, a standard xeno-tissue media for culturing stem cells, and better than matrigel, a widely used feeder-free substrate.

The present disclosure further provides that the substrates can be employed with other proteins in a suitable cell culture medium to promote colony formation. In certain embodiments, the proteins include, but are not limited to, serum, fibronectin, laminin, vitronectin, collagen, and any combination thereof. In some embodiments, the substrates employ integrin engagement with adsorbed vitronectin to promote colony formation.

In certain embodiments, the suitable culture medium comprises soluble factors that enhance propagation of the stem cells. Examples of the suitable culture medium include, but are not limited to, MEFs-conditioned medium or mTeSR medium. In certain embodiments, this disclosure provides that the propagated stem cells cultured using the device and methods express pluripotency markers of human stem cells after at least 10 passages, including but not limited to Tra1-60, Nanog, Oct4, Sox2, and SSEA4.

A wide range of polymers can be used for the array synthesis such that the desired surface chemical ion signatures and optimal surface properties are attained, examples of which include, but are not limited to, the acrylic family of polymers such as polymers and copolymers of acrylic and methacrylic esters and other derivatives. In one aspect, for example, suitable monomers for preparing these polymers include the acrylate-, diacrylate-, and methacrylate-based monomers. Diacrylate compounds work particularly well. An acrylate-type moiety in such monomers can be linked, for example to another acrylate, olefin, hydroxyl, or other functionality by a linker. Examples of linker moieties include, but are not limited to, oligomeric oxy(alkandiyl) linker of various lengths (including —OCH₂CH₂—), cycloalkyl linkers, aryl linkers, fused or bicyclic hydrocarbyl linker groups, and the like, all of which are encompassed in this disclosure. Monomers can be further functionalized with, for example, halide, ether, hydroxyl, and other such groups, including substitutions at various positions along the linker.

The substrates of the present disclosure can be virgin bacterial grade polystyrene and/or ultralow attachment surfaces treated with UV/ozone under a photomask. In certain embodiments, the substrates comprising polymers or polymer arrays are generated from monomers with high acrylate content and polymerized with a UV source. In certain embodiments, the polymer is selected from a virgin bacterial grade polystyrene and/or ultralow attachment surface treated with UV/ozone under a photomask.

Methods of in vitro culturing, propagation, and maintenance of human stem cells using the device and the cell culture system of the present disclosure are also provided. The disclosed method comprises culturing the human stem cells in a suitable culture medium on the device of the present disclosure comprising a substrate adapted for culturing stem cells, and is characterized by a SIMS ion signature corresponding to a predetermined ion signature correlated with a desired behavior in the stem cells, and wherein the substrate is untreated or treated to generate the predetermined SIMS ion signature.

As contemplated in the present disclosure, the stem cells used herein include pluripotent, multipotent, oligopotent and totipotent stem cells from human and animal tissues. For example, the human stem cells can include human embryonic stem cells (hESCs) and human induced pluripotent stem cells (hiPSCs). The device and culture system of this disclosure can be used for clonal expansion and maintenance of stem cells, as well as for somatic cell reprogramming to generate patient-specific hiPS cells, for gene targeting of stem cells, and for direct differentiation of stem cells into ectodermal, mesodermal, and endodermal lineages, and for further terminal cellular differentiation.

The present disclosure provides devices and methods for culturing stem cells comprising the substrate exhibiting the disclosed ion signatures and other surface properties that improve the stem cell culturing efficiency by at least 10%, 20%, 30%, 40%, 50%, 75%, 100%, 150%, 200%, 300%, 500%, 750%, 1000%, 1500%, 2000%, or more, as compared to the devices and methods for culturing stem cells comprising substrates that lack the disclosed ion signatures and other surface properties.

### BRIEF DESCRIPTION OF THE DRAWINGS

FIGS. 1a-b provide biomaterial array design for clonal growth. a) monomers used for array synthesis were classified into two categories: “major” monomers that constitute >50% of the reactant mixture and “minor” monomers that constitute <50% of the mixture. Sixteen (16) major monomers were named numerically (1-16), and six (6) minor monomers were labeled alphabetically (A-F); b) shows an example of thirty-six (36) different combinations (ratios are in v/v) for the major monomer 1 with all 6 different minor monomers. Same combinations for major monomers 2-16 with all 6 different minor monomers were also provided. All monomers were combined in a combinatorial fashion to generate a diverse polymer array.

FIG. 2 shows a photograph showing the polymer microarray with sixteen (16) polymer spots to illustrate dimension and separation.

FIG. 3 illustrates mapping cell behavior to surface chemistry using secondary arrays. Time-of-flight secondary ion mass spectrometry (ToF-SIMS or simply SIMS) spectra of homopolymers 1 (FIG. 3a) and 16 (FIG. 3b) indicating that the surface chemistry cannot be necessarily predicted from the monomer chemistry. Arrows delineate higher intensities of hydrocarbon secondary ions (C₃H₅⁺, C₃H₇⁺) and ester ions (C₂H₃O⁺) in the homopolymer 1 spectra. In contrast, higher intensity of ethylene glycol ions (C₂H₅O⁺) and propylene glycol ions (C₃H₇O⁺) were observed in the homopolymer 16 spectra.

FIG. 4 illustrates surface chemical analysis of the 16 homopolymers using principal component analysis. FIG. 4a provides a map of the 16 homopolymers generated from the major monomers in FIG. 1a, according to their loadings along the two major principal components (“PC”), PC1 and PC2, from principal component analysis of their ToF-SIMS spectra. Each polymer contains six repeats. Polymers with propylene/ethylene glycol moieties are labeled. Note that the glycol moiety containing polymers 3, 16 and 6 differs from other glycol moiety containing polymers 9, 1, 2, and 11 in their PC1 and PC2 loadings. FIG. 4b illustrates ion loadings of the various ToF-SIMS spectra in each principal component. The PC2 loading has several secondary ions that help separate the glycol containing moieties 3, 16, and 6 from the other glycol moiety containing polymers.

FIG. 5 illustrates short- and long-term feeder-free culture on “hit” polymer arrays and efficiencies of various culture systems to support undifferentiated growth of dissociated hES cells. Two media conditions were used, labeled at the bottom: mEF-conditioned media (MEF-CM) or chemically defined media (mTeSR1). Several combinations of substrate and protein coating were used in conjunction with these media. Three substrates consisted of tissue culture polystyrene (TCPS), hit polymer 9 (“9”; see FIG. 1a for monomer structure), and hit polymer 15A-30% (“15A”; see FIG. 1a for monomer structures). Four protein coatings consisted of matrigel, bovine serum, human serum, and human vitronectin. Further, mEFs on gelatin-coated TCPS in regular hES media was also used. In each condition, efficiencies were calculated as the number of SSEA-4+ and Oct4+ colonies seen on day 7 normalized to the number of cells attached on day 1. This metric specifically reflects the ability of substrates to promote undifferentiated clonal cell growth after correcting for any differences in initial cell attachment.

FIG. 6 compares material properties on the primary and secondary arrays. FIG. 6a provides forty-eight (48) different combinations for the major and minor monomers for the newly designed secondary array. Monomer structure are shown in FIG. 1a. FIG. 6b. provides water contact angles of all 496 polymers in the primary array and the newly designed 48 secondary polymer array. Similar coverage of properties was achieved with the secondary array. FIG. 6c illustrates colony formation frequency versus water contact angle for all polymers in the secondary array. Nonlinear regression indicates an optimum at 67° C. FIG. 6d illustrates colony formation on polymers in both the primary and secondary arrays versus water contact angle. In comparing the primary and secondary array results, no statistically significant differences were observed (analysis of variance Tukey-Kramer test, p<0.05). As designated by the asterisks (*), results on polymers in the extreme WCA bins of 30-45° C. and 90-105° C. were statistically different from the moderate WCA bins of 60-90° C. (analysis of variance Tukey-Kramer test, p<0.05).

FIG. 7 illustrates variance in water contact angle measurements. FIG. 7a, Materials with similar reduced indentation elastic modulus could have very different WCAs. Some examples are underlined. FIG. 7b, Measurement error was low (error bars, s.e.=0.9-6.9%), as indicated by very consistent results in WCA measurements on 6 replicates of 16 homopolymers.

FIG. 8 illustrates surface chemical analysis using multivariate partial least squares (PLS) model of the ToF-SIMS data. Ions, with the highest regression coefficients, “α”, were identified as supporting (α>0) or inhibiting (α<0) hES cell colony formation.

FIG. 9 illustrates αᵥβ₅ integrin blocking reduces initial adhesion of hES cells on hit polymers. The fraction of adhered cells after 24 hr of culture on hit polymer arrays coated with either human serum (HS) or human vitronectin (Vn) and with the specified integrin blocking antibody are plotted. The cell numbers shown here are an average of 24 replicates of the following hit polymers: 15, 15B-10%, 15B-20%, 15B-25%, 15D-10, and 15D-20%. β1 Blocking had minimal effect either alone or in combination with αυβ₅ blocking, whereas αυβ₅ blocking reduced adhesion by ˜50%.

FIG. 10 illustrates integrin-blocking cell behavior on UV/ozone-patterned polystyrene is similar to hit polymers. hES Cells were single cell seeded on UV/ozone-patterned polystyrene dishes and then grown in the presence of various blocking antibodies for 24 hrs in mTESR1, fully-defined media. Cell adhesion is blocked only by the αᵥβ₅ integrin (vitronectin receptor) blocking antibody and not the β1 blocking antibody. Dishes were pre-incubated with media with 20% human serum.

FIG. 11 provides the composition of mTeSR1 chemically defined media. Composition is identical to the total animal-free medium, TeSR, except for the use of bovine serum albumin and recombinant FGF. Key growth factors and serum albumin components are bolded.^{27,28}

FIG. 12 illustrates surface chemical analysis using multivariate partial least squares (PLS) model of vitronectin-coated secondary array ToF-SIMS data. FIG. 12a, Predicted ESC colony formation probability from ToF SIMS analysis of vitronectin coated secondary array using PLS. Labels indicate the polymer composition, as listed in FIG. 1a. Note that this prediction does not predict behavior as well as using the spectra from the bare polymers. This result suggests that there may be something more in the serum that interacts with the polymers to enhance colony formation. FIG. 12b, Ions, with the highest regression coefficients, “α”, were identified as supporting (α>0) or inhibiting (α<0) hES colony formation. While ToF-SIMS can identify monomers that help vitronectin adsorption, these monomers are not necessarily beneficial for colony formation.

FIG. 13 illustrates surface chemical analysis of the vitronectin-coated secondary array using principal component analysis. FIG. 13A, Map of the polymers generated from the major monomers listed in FIG. 1a, according to their loadings along the two major principal components, PC1 and PC2, from principal component analysis of their ToF-SIMS spectra. Each polymer contains six repeats. FIG. 13B, Ion loadings of the various ToF-SIMS spectra in each principal component. Polymers with higher PC1 values has more nitrogen containing ions from vitronectin.

FIG. 14 provides characteristic ions supporting or inhibiting clonal growth on the UV/ozone treated polystyrene using PLS-analysis on the ToF-SIMS data.

### DETAILED DESCRIPTION OF THE INVENTION

Among other things, this disclosure applies a device and methods of use thereof to support the culturing and propagation of stem cells. The present disclosure provides that the chemical ion signature, as well as optimal surface energy (e.g., water contact angle) and the confined environment created by micrometer scale spots, are important to support culturing of stem cells. Therefore, this disclosure provides that the unique combination defined by the chemical ion signatures, the optimal surface energy properties, and confined environments can effectively support culturing, propagating, maintaining, and differentiating of stem cells as well as reprogramming somatic cells into stem cells. Further, this disclosure provides that the chemical ion signature as determined by the secondary ion mass spectrometry method and conditions described herein, can be correlated with a specific and desired cell behavior, such as culturing human pluripotent stem cells, and a selected substrate that displays this ion signature can be adapted for culturing stem cells, whether untreated or treated (e.g. with UV/ozone oxidation) to generate the chemical ion signature.

The present disclosure provides a device, and methods of use thereof, comprising a substrate having significant chemical ion signatures and providing optimal surface energies that support culturing, expansion, differentiation of stem cells, as well as reprogramming somatic cells into stem cells, preserve a normal karyotype, and maintain differentiation capacity after prolonged cell culture. The substrates described herein provide a unique chemically defined, xeno-free, feeder-free system to support efficient clonal growth of stem cells, including human pluripotent stem cells.

As used herein, a “stem cell” means a cell of human or animal origin that can produce daughter cells that have different, more restricted properties, and therefore, is not terminally differentiated. Stem cells include pluripotent stem cells, which can form cells of any of the body's tissue lineages: mesoderm, endoderm and ectoderm. Therefore, for example, stem cells can be selected from a human embryonic stem (ES) cell; a human inner cell mass (ICM)/epiblast cell; a human primitive ectoderm cell, a human primitive endoderm cell; a human primitive mesoderm cell; and a human primordial germ (EG) cell. Stem cells also include multipotent stem cells, which can form multiple cell lineages that constitute an entire tissue or tissues, such as but not limited to hematopoetic stem cells or neural precursor cells. Stem cells also include totipotent stem cells, which can form an entire organism. In some embodiments, the stem cell is a partially differentiated or differentiating cell. In some embodiments, the stem cell is an induced pluripotent stem cell (iPSC), which has been reprogrammed or de-differentiated. Stem cells can be obtained from embryonic, fetal or adult tissues. The stem cells of the present disclosure can be derived in vivo or in vitro using any method known to those of skill in the art at the present time or later discovered.

In certain embodiments of this disclosure, the stem cell culture is an essentially homogenous cell culture with respect to a desired characteristic, such as but not limited to karyotype, cell marker expression pattern, or cellular differentiation potential. In one embodiment, the essentially homogenous cell culture consists of cells that have a normal karyotype. For example, it is contemplated that in such karyotypically essentially homogenous cell cultures, greater than 50%, 60%, 70%, 75%, 80%, 85%, 90%, 91%, 92%, 93%, 94%, 95%, 96%, 97%, 98%, or 99% of metaphases examined will display a normal karyotype. In certain embodiments, the normal karyotype can be evident after the cells have been dissociated to an essentially single cell culture for greater than 5, 10, 15, 20, or more passages.

In one embodiment of the present invention, the stem cell culture is stable in culture. As used herein, the terms “stable” and “stabilize” refer to the differentiation state of a cell or cell line. When a cell or cell line is stable in culture, it will continue to proliferate without significant differentiation over multiple passages, and in some cases indefinitely, in culture. Therefore, certain stem cells in an essentially homogenous stem cell culture are preferably of the same differentiation state, and when the cells divide, typically yield cells of the same cell type or yield cells of the same differentiation state. In other embodiments, the devices and methods of the present invention are intended to cause the stem cells to differentiate or partially differentiate into daughter cells with more restricted properties, and thus create essentially homogenous differentiated cell cultures.

In other embodiments, the cell culture environment comprises seeding the stem cells on a substrate adapted for culturing stem cells in an adherent culture. As used herein, the terms “seeded” and “seeding” refer to any process that allows a cell be cultured in adherent culture. As used herein, the term “adherent culture” refers to a cell culture device and system whereby cells are cultured on a solid substrate as described herein. The cells may or may not tightly adhere to the solid surface or to the substrate.

In certain embodiments, the substrates of the device are characterized by a secondary ion mass spectrometry (SIMS) ion signature corresponding to a predetermined ion signature correlated with a desired behavior in the stem cells. The substrate of this disclosure can be untreated or treated to generate the predetermined SIMS ion signature. In certain embodiments, the substrate comprises a polymer or a polymer array comprising at least 10 polymer domains distributed on a support. U.S. Patent Application Publication No. 2005/0019747, which is incorporated by reference in its entirety, describes a nanoliter-scale synthesis of arrayed biomaterials and screening thereof. In certain embodiments, the substrate can comprise a polymer which is characterized by a SIMS ion signature comprising at least one of the three most intense ion peaks selected from a hydrocarbon ion having no tertiary carbon atoms, a cyclic hydrocarbon ion, or an oxygen-containing ion derived from an ester.

In certain embodiments, the substrate comprises an acrylate-based polymer or copolymer having a SIMS ion signature comprising at least one of the three most intense ion peaks selected from a C₁₋₄ hydrocarbon ion having no tertiary carbon atoms, a cyclic hydrocarbon ion, an oxygen-containing ion derived from an ester, O⁻, and OH⁻. In other embodiments, the SIMS ion signature comprises at least one of the three most intense ion peaks selected from O⁻, C₂H⁻, OH⁻, CHO₂⁻, C₂H₃⁻, C₃H₅⁺, C₄H⁻, C₁₀H₁₁O⁻, CH⁻, C₃H₃⁻, C₃H₇⁺, C₂H₅O⁺, and C₂H₃O⁺. In yet other embodiments, the SIMS ion signature comprises a base peak selected from O⁻, C₂H⁻, OH⁻, CHO₂⁻, C₂H₃⁻, C₃H₅⁺, C₄H⁻, C₁₀H₁₁O⁻, CH⁻, C₃H₃⁻, C₃H₇⁺, C₂H₅O⁺, and C₂H₃O⁺, and at least one of the two subsequent ions according to peak intensity selected from O⁻, C₂H⁻, OH⁻, CHO₂⁻, C₂H₃⁻, C₃H₅⁺, C₄H⁻, C₁₀H₁₁O⁻, CH⁻, C₃H₃⁻, C₃H₇⁺, C₂H₅O⁺, and C₂H₃O⁺. In still other embodiments, the SIMS ion signature comprises a base peak selected from O⁻, C₂H⁻, OH⁻, CHO₂⁻, C₂H₃⁻, C₃H₅⁺, C₄H⁻, C₁₀H₁₁O⁻, CH⁻, C₃H₃⁻, C₃H₇⁺, C₂H₅O⁺, and C₂H₃O⁺. In certain embodiments, the acrylate-based polymer or copolymer of the present disclosure has a SIMS ion signature comprising the three most intense ion peaks selected from an ion other than CN⁻, C₂H₇O⁺, C₄H₉⁺, C₂H₆N⁺, C₃H₃O₂⁻, C₃H₈N⁺, C₅H₉⁺, C₅H₁₁⁺, CNO⁻, and C₃H₇O⁺. In certain embodiments, the acrylate-based polymer or copolymer of the present disclosure has a SIMS ion signature comprising the base peak selected from an ion other than CN⁻, C₂H₇O⁺, C₄H₉⁺, C₂H₆N⁺, C₃H₃O₂⁻, C₃H₈N⁺, C₅H₉⁺, C₅H₁₁⁺, CNO⁻, and C₃H₇O⁺.

The various ion signatures that are inclusive and exclusive of certain ions are intended to be disclosed individually or together in any combination, as basic chemical principles allow. For example, one combination of inclusive and exclusive ions that together can constitute an ion signature is provided as follows. In some embodiments, the SIMS ion signature comprises at least one of the three most intense ion peaks selected from O⁻, C₂H⁻, OH⁻, CHO₂⁻, C₂H₃⁻, C₃H₅⁺, C₄H⁻, C₁₀H₁₁O⁻, CH⁻, C₃H₃⁻, C₃H₇⁺, C₂H₅O⁺, and C₂H₃O⁺, in combination with the feature that the SIMS ion signature comprising the base peak selected from an ion other than CN⁻, C₂H₇O⁺, C₄H₉⁺, C₂H₆N⁺, C₃H₃O₂⁻, C₃H₈N⁺, C₅H₉⁺, C₅H₁₁⁺, CNO⁻, and C₃H₇O⁺.

In other embodiments, the substrate comprises a styrene-based polymer having a SIMS ion signature comprising at least one of the three most intense ion peaks selected from a C₂₋₆ hydrocarbon ion having no tertiary carbon atoms, a cyclic hydrocarbon ion, or an oxygen-containing ion derived from an ester. In certain embodiments, the SIMS ion signature comprises at least one of the three most intense ion peaks characterized by a carbon-to-hydrogen atomic ratio of less than 1. In yet other embodiments, the SIMS ion signature comprises at least two of the three most intense ion peaks characterized by a carbon-to-hydrogen atomic ratio of less than 1.

In yet other embodiments, the SIMS ion signature comprises at least one of the three most intense ion peaks selected from C₂H₄O⁺, C₆H₉O⁺, C₃H₃O⁺, C₂H₃⁺, C₆H₁₁⁺, C₂H₅⁺, C₂H₃O⁺, C₅H₇O⁺, and C₃H₅⁺. In some embodiments, the SIMS ion signature comprises a base peak selected from C₂H₄O⁺, C₆H₉O⁺, C₃H₃O⁺, C₂H₃⁺, C₂F⁻, C₆H₁₁⁺, C₂H₅⁺, C₂H₃O⁺, C₅H₇O⁺, and C₃H₅⁺, and at least one of the two subsequent ions according to peak intensity selected from C₂H₄O⁺, C₆H₉O⁺, C₃H₃O⁺, C₂H₃⁺, C₂F⁻, C₆H₁₁⁺, C₂H₅⁺, C₂H₃O⁺, C₅H₇O⁺, and C₃H₅⁺.

In certain embodiments, the styrene-based polymer of the present disclosure has a SIMS ion signature comprising at least one of the three most intense ion peaks selected from an ion other than C₇H₇⁺, CHO₂⁻, C₉H₉⁺, O⁻, C₇H₅O⁺, C₉H₇⁺, C₆H₅⁺, C₂H⁻, C₈H₇⁺, and C₇H₇O⁺. In certain embodiments, the styrene-based polymer of the present disclosure has a SIMS ion signature comprising the base peak selected from an ion other than C₇H₇⁺, CHO₂⁻, C₉H₉⁺, O⁻, C₇H₅O⁺, C₉H₇⁺, C₆H₅⁺, C₂H⁻, C₈H₇⁺, and C₇H₇O⁺.

Similarly, for the styrene-based polymers as in any substrate material, various ion signatures that are inclusive and exclusive of certain ions are intended to be disclosed individually or together in any combination, as basic chemical principles allow. For example, one combination of inclusive and exclusive ions that together can constitute an ion signature for the styrene-based polymers is provided as follows. In some embodiments, a SIMS ion signature can comprise at least one of the three most intense ion peaks selected from C₂H₄O⁺, C₆H₉O⁺, C₃H₃O⁺, C₂H₃⁺, C₆H₁₁⁺, C₂H₅⁺, C₂H₃O⁺, C₅H₇O⁺, and C₃H₅⁺, in combination with the feature that the SIMS ion signature can comprise at least one of the three most intense ion peaks selected from an ion other than C₇H₇⁺, CHO₂⁻, C₉H₉⁺, O⁻, C₇H₅O⁺, C₉H₇⁺, C₆H₅⁺, C₂H⁻, C₈H₇⁺, and C₇H₇O⁺.

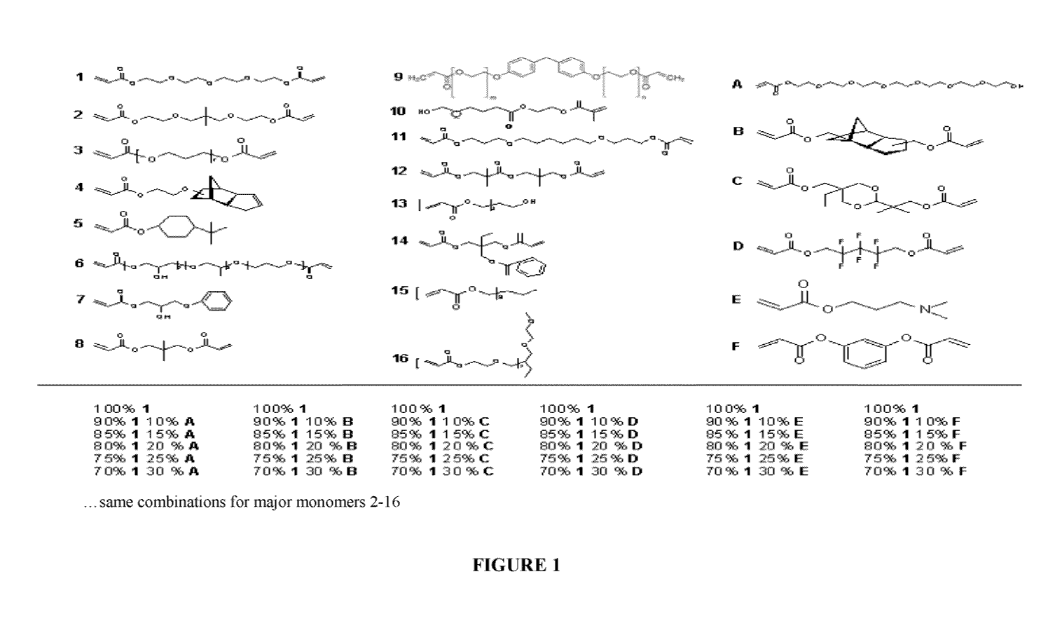

In certain embodiments of the present disclosure, at least two suitable polymers were selected and they were used to fabricate arrays. FIGS. 1a-b provide polymer array design and system for clonal growth comprising major and minor monomers mixed in v/v ratios. All monomers were combined in a combinatorial fashion to generate a diverse polymer array. The diverse polymer array validated the results from primary screening, and further confirmed their capacity to maintain pluripotency of human stem cells, preserve normal karyotype, and maintain full differentiation capacity after prolonged cell culture. In addition, the efficacy of polymer spots to support single cell growth of human pluripotent stem cells were found to be similar to MEFs, a standard to culture hESCs, and better than matrigel, a widely used feeder free substrate.

In further aspects, this disclosure provides that other properties including surface wettability and/or optimal surface energy, e.g., water contact angle, and the confined environments created by the micrometer scale spots, in particular their periphery, are important to support culturing, expansion, and differentiation of human multipotent and pluripotent stem cells as well as reprogramming of somatic cells. The present disclosure thus contemplates that the unique combination defined by the optimal surface properties and confined environments can effectively support culturing, expansion, and differentiation of human multipotent and pluripotent stem cells as well as reprogramming of somatic cells.

In certain embodiments, the device of the present disclosure comprises a substrate comprising an array of at least 10 polymer domains distributed on a support, and each domain has a moderate wettability with a water contact angle (WCA) of about 45° to 90° C. In some embodiments, each domain has a moderate wettability with a water contact angle (WCA) of about 55° to 80° C. Alternatively, each domain can have a moderate wettability with a water contact angle (WCA) of about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C., or about 80° C.

In certain embodiments, the array of polymer domains comprises a repeating microenvironment array adapted for culturing and expansion of human multipotent and pluripotent stem cells. Each microenvironment comprises the peripheral aspect of each microspots having a major axis in a range of about 1 μm to 1000 μm. The term “major axis” is used to describe both regularly-shaped microspots, for example, circular microspots in which the major axis is the diameter, and those that are irregularly shaped, where the major axis corresponds to the greatest linear distance from one end of the microspot or object to another end, that is, its longest diameter. In certain embodiments, the major axis of each polymer microspots is in a range of about 10 μm-500 μm; alternatively in a range of about 100 μm-450 μm; or alternatively, in a range of about 200 μm-400 μm. In another aspect, each polymer microspots can have a major axis of about: 10 μm, 20 μm, 30 μm, 40 μm, 50 μm, 60 μm, 70 μm, 80 μm, 90 μm, 100 μm, 110 μm, 120 μm, 130 μm, 140 μm, 150 μm, 160 μm, 170 μm, 180 μm, 190 μm, 200 μm, 210 μm, 220 μm, 230 μm, 240 μm, 250 μm, 260 μm, 270 μm, 280 μm, 290 μm, 300 μm, 310 μm, 320 μm, 330 μm, 340 μm, 350 μm, 360 μm, 370 μm, 380 μm, 390 μm, 400 μm, 410 μm, 420 μm, 430 μm, 440 μm, 450 μm, 460 μm, 470 μm, 480 μm, 490 μm, or 500 μm.

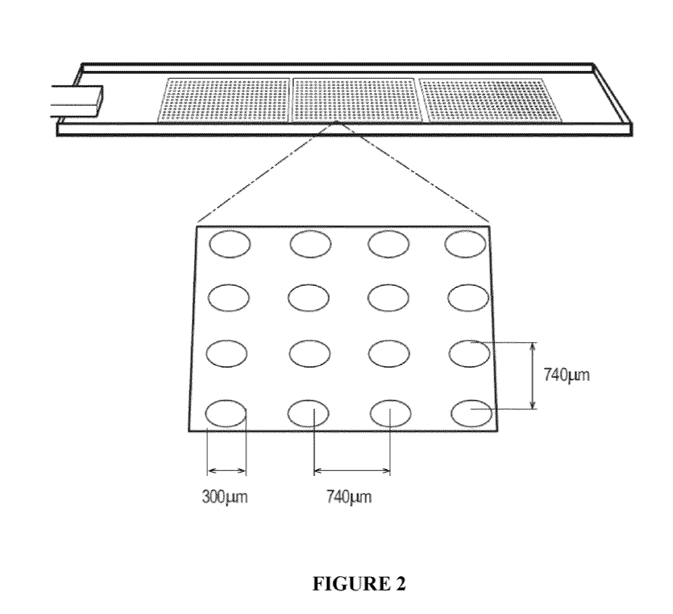

Those of skill in the art can readily ascertain the water contact angle of a substrate, or review the published values. In particular, the Examples herein provide such guidance of operable embodiments. In certain embodiments, a confined microenvironment is exemplified where each substrate microspot of the present disclosure has a major axis in a range of 1 μm-1,000 μm, preferably, 10 μm-500 μm, more preferably, about 300 μm. FIG. 2 provides a photograph showing the polymer microarray with sixteen (16) polymer spots to illustrate dimension and separation. In the disclosure and claims, the polymer spots themselves may be referred to as a substrate, for example a substrate adapted for culturing stem cells, while the material on which the polymer spots are situated, for example a glass slide, may be referred to as a support. The material such as a glass slide may be referred to herein as a “support” for the polymer spots, as the context provides.

In certain embodiments, the microspots are discrete and separate and in other embodiments, the microspots can overlap to varying degrees. In alternative embodiments, the microspots can be any shape, in addition to being round, in particular to maximize the peripheral microenvironment, such as in a star-shape, jagged-edge or scaffold pattern. In alternative embodiments, the microenvironment is created using substrate exhibiting the desired surface property (WCA) but shaped into contiguous planar or non-planar textured surfaces. In certain embodiments, the array contains at least 10, 20, 25, 50, 75, 100, 200, 500, 1000, or more microspots of the same substrate. In certain embodiments, more than one substrate or other modifiers or agents can be used to make the microspots.

The present disclosure further provides that the substrates are employed with other proteins in a suitable cell culture medium to promote colony formation. In certain embodiments, the proteins include, but are not limited to, serum, fibronectin, laminin, vitronectin, collagen, and any combination thereof. The suitable culture medium contemplated in the present disclosure includes any cell culture medium suitable for culturing human multipotent and pluripotent stem cells and may comprise soluble factors that enhance propagation of human pluripotent stem cells.

Examples of the suitable culture medium include, but not limited to, MEFs-conditioned medium or mTeSR medium. In some embodiments, the substrates employ integrin engagement with adsorbed vitronectin to promote colony formation. According to some aspects, this disclosure provides that the propagated human pluripotent stem cells on the substrate microspot express markers unique for the human pluripotent stem cells after at least 10 passages. Such unique human pluripotent stem cell markers include, but not limited to Tra1-60, Nanog, Oct4, Sox2, and SSEA4. Preferably, the colony is essentially homologous, such that greater than 50%, 60%, 70%, 75%, 80%, 85%, or 90% of the propagated human pluripotent stem cells express the marker, more preferably, greater than 91%, 92%, 93%, 94%, 95%, 96%, 97%, 98%, of the cells of the colony express the marker, and still more preferably, greater than 99% of the cells of the colony express the marker.

The substrate for the microspots, typically referred to as a “support,” comprises any suitable support material including for example glass and silanized glass. A wide range of substrates can be used for the array synthesis such that the desired surface energy is attained, examples of which include, but are not limited to, the acrylic family of polymers such as polymers and copolymers of acrylic and methacrylic esters and other derivatives. In one aspect, for example, suitable monomers for preparing these polymers include the acrylate-, diacrylate-, and methacrylate-based monomers. Diacrylate compounds work particularly well. An acrylate-type moiety in such monomers can be linked, for example to another acrylate, olefin, hydroxyl, or other functionality by a linker. Examples of linker moieties include, but are not limited to, oligomeric oxy(alkandiyl) linker of various lengths (including —OCH₂CH₂—), cycloalkyl linkers, aryl linkers, fused or bicyclic hydrocarbyl linker groups, and the like, all of which are encompassed in this disclosure. Monomers can be further functionalized with, for example, halide, ether, hydroxyl, and other such groups, including substitutions at various positions along the linker. Specific examples of suitable monomers are illustrated in FIG. 1a, along with a listing of some polymers and copolymers that can be prepared using these monomers.

The substrates of the present disclosure can be UV/ozone-treated virgin bacterial grade polystyrene. In certain embodiments, the substrates comprising polymers that are generated from monomers with high acrylate content and polymerized with a UV source. In certain embodiments, the substrate comprises polystyrene that is selected from a UV/ozone-treated virgin bacterial grade polystyrene.

Unless otherwise noted, the terms used herein are to be understood according to conventional usage by those of ordinary skill in the relevant art. In addition to the definitions of terms provided below, definitions of common terms in molecular biology may also be found in Rieger et al., 1991 Glossary of genetics: classical and molecular, 5th Ed, Berlin: Springer-Verlag; in Current Protocols in Molecular Biology, F. M. Ausubel et al., eds., Current Protocols, a joint venture between Greene Publishing Associates, Inc. and John Wiley & Sons, Inc., (latest Supplement); and in Current Protocols in Cell Biology, J. S. Bonifacino et al., Eds., Current Protocols, John Wiley & Sons, Inc. (latest Supplement). To the extent that any definition or usage provided by any of the references or any documents incorporated by reference conflicts with the definition or usage provided herein, the definition or usage provided herein controls. It is to be understood that as used in the specification and in the claims, “a” or “an” can mean one or more, depending upon the context in which it is used. Thus, for example, reference to “a cell” can mean that at least one cell can be utilized.

Methods of in vitro culturing and expansion of human multipotent and pluripotent stem cells using the device and/or the cell culture system of the present disclosure are also provided. For example, the disclosed method comprises culturing the human multipotent and pluripotent stem cells in a suitable culture medium on the present device comprising a substrate adapted for culturing stem cells, and characterized by a secondary ion mass spectrometry (SIMS) ion signature corresponding to a predetermined ion signature correlated with a desired behavior in the stem cells. In certain embodiments, the substrates used in the disclosed method comprise polymers that is characterized by the SIMS ion signature comprising at least one of the three most intense ion peaks selected from a hydrocarbon ion having no tertiary carbon atoms, a cyclic hydrocarbon ion, or an oxygen-containing ion derived from an ester, and the device is adapted for culturing human multipotent and pluripotent stem cells.

The step of culturing the human stem cells with the suitable medium in the presence of a suitable substrate to support culturing, expansion, and differentiation of human multipotent and pluripotent cells as well as reprogramming of somatic cells can be conducted in any suitable manner. The present disclosure also provides that the present device and methods can be used for clonal expansion of human multipotent and pluripotent stem cells, as well as for somatic cell reprogramming to generate patient-specific human induced pluripotent cells, for gene targeting of human embryonic stem cells, and for direct differentiation of human embryonic stem cells into ectodermal, mesodermal, and endodermal fates.

Therefore, the present disclosure provides a number of substrates that can be employed to regulate a range of cell behaviors for tissue engineering applications, including adhesion, proliferation, differentiation, and reprogramming. In certain aspects, the present disclosure provides that it is the chemical ion signature along with the surface properties of the substrates that determine the culturing and propagation of human multipotent and pluripotent stem cells. Thus, this disclosure contemplates any substrates having the desired chemical ion signature and surface properties.

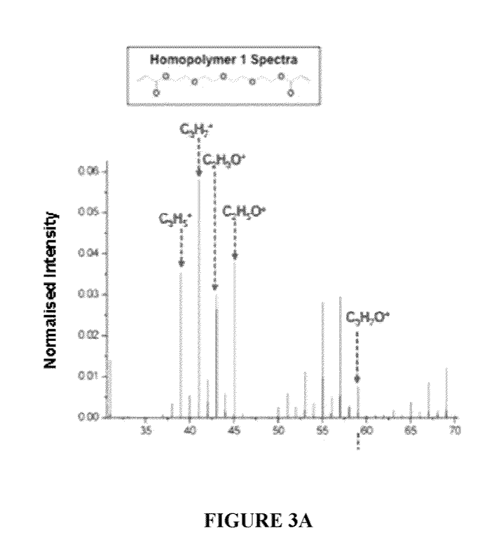

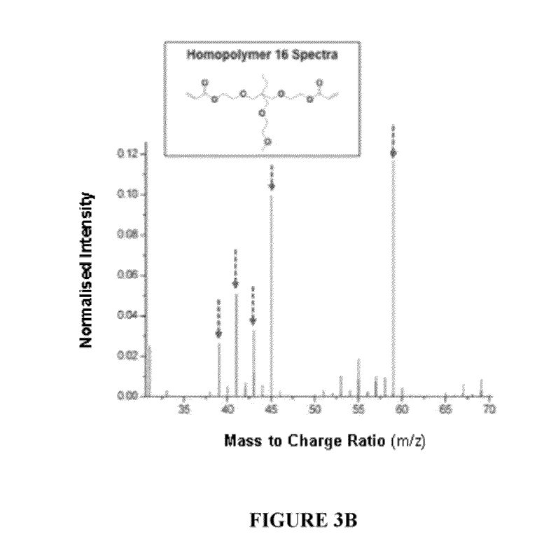

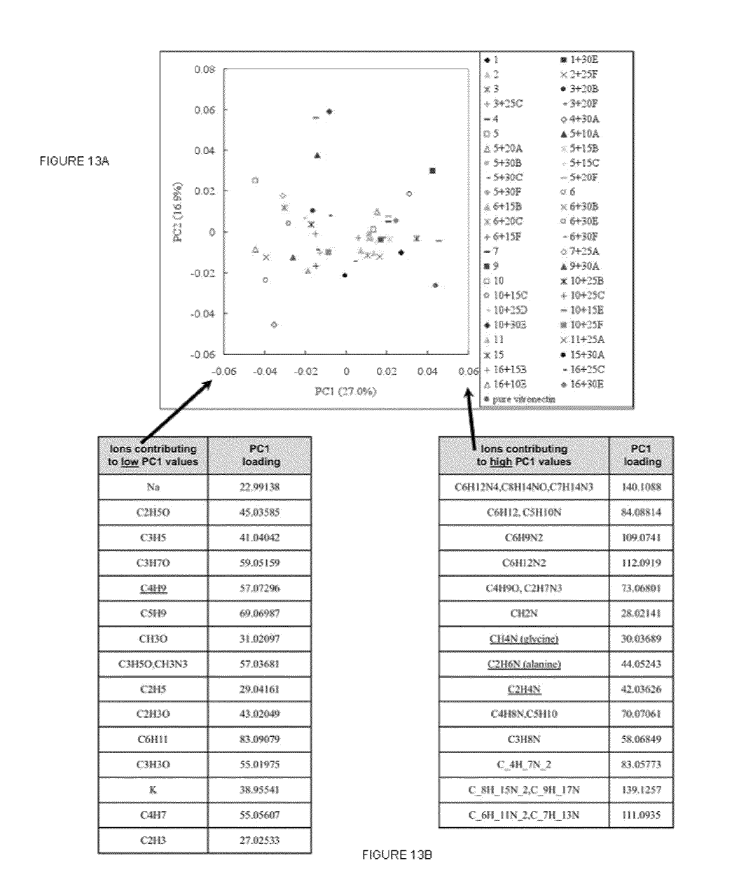

In certain embodiments, suitable ion signatures contemplated in the present disclosure are presented in FIGS. 3, 4, 8, 12, 13, and 14. In particular, in certain embodiments, the suitable ion signature supporting cell growth includes O⁻, C₂H⁻, OH⁻, CHO₂⁻, C₂H₃⁻, C₃H₅⁺, C₄H⁻, C₁₀H₁₁O⁻, CH⁻, C₃H₃⁻, C₃H₇⁺, C₂H₅O⁺, and C₂H₃O⁺. In yet other embodiments, the suitable ion signature supporting cell growth includes C₂H₄O⁺, C₆H₉O⁺, C₃H₃O⁺, C₂H₃⁺, C₂F⁻, C₆H₁₁⁺, C₂H₅⁺, C₂H₃O⁺, C₅H₇O⁺, and C₃H₅⁺.

The present disclosure provides devices and methods for culturing stem cells comprising the substrate exhibiting the disclosed ion signatures and other surface properties that improve the efficiency of stem cell culturing by at least 10%, 20%, 30%, 40%, 50%, 75%, 100%, 150%, 200%, 300%, 500%, 750%, 1000%, 1500%, 2000%, or more, as compared to the devices and methods for culturing stem cells comprising substrates that lack the disclosed ion signatures and other surface properties.

Throughout this application, various publications are referenced. The disclosures of all of these publications and those references cited within those publications in their entireties are hereby incorporated by reference into this application in order to more fully describe the state of the art to which this disclosure pertains. Nothing herein is to be construed as an admission that the inventors are not entitled to antedate such disclosure by virtue of prior invention.

To the extent that any definition or usage provided by any document incorporated herein by reference conflicts with the definition or usage provided herein, the definition or usage provided herein controls.

Unless indicated otherwise, when a range of any type is disclosed or claimed, it is intended that the recited range is inclusive of the upper and lower limits of the range. Therefore, the term “in a range” and similar terms are intended to mean from the lower limit of the range to the upper limit of the range, inclusive. Further, and unless indicated otherwise, when a range of any type is disclosed or claimed, for example a range of the number of carbon atoms, diameters of major axis sizes, molar ratios, temperatures, and the like, it is intended to disclose or claim individually each possible number that such a range could reasonably encompass, including any sub-ranges encompassed therein. For example, when describing a range of the number of carbon atoms, each possible individual integral number and ranges between integral numbers of atoms within that broadly disclosed range are encompassed therein. Thus, by disclosing a C₁ to C₆ hydrocarbyl group, or a C₁₋₆ hydrocarbyl group, alternatively described as a hydrocarbyl group having from 1 to 6 carbon atoms or “up to” 6 carbon atoms, Applicants' intent is to recite that the hydrocarbyl group can have 1, 2, 3, 4, 5, or 6 carbon atoms, and these methods of describing such a group are interchangeable. Similarly, when applicants disclose a range of diameters or major axis distances or any other measurement, each possible number that such a range could reasonably encompass is included in this disclosure, usually to values within the range with one significant digit more than is present in the end points of a range, unless otherwise indicated. For example, by disclosing that a major axis can have a range of 50 μm-70 μm, such a disclosure is intended to be equivalent to the disclosure that the major axis can be 50 μm, 51 μm, 52 μm, 53 μm, 54 μm, 55 μm, 56 μm, 57 μm, 58 μm, 59 μm, 60 μm, 61 μm, 62 μm, 63 μm, 64 μm, 65 μm, 66 μm, 67 μm, 68 μm, 69 μm, or 70 μm, including any ranges or combinations of ranges between these recited numbers, inclusive. Therefore, Applicants also intend for the disclosure of a range to reflect, and be interchangeable with, disclosing any and all sub-ranges and combinations of sub-ranges encompassed therein. By way of example, Applicants' disclosure of a range of 50 μm-70 μm is intended to literally encompass 50 μm-58 μm, 55 μm-63 μm, 60 μm-70 μm, and any combinations of such ranges, and so forth. Accordingly, Applicants reserve the right to proviso out or exclude any individual members of any such group, including any sub-ranges or combinations of sub-ranges within the group, if for any reason Applicants choose to claim less than the full measure of the disclosure, for example, to account for a reference that Applicants are unaware of at the time of the filing of the application.

In any application before the United States Patent and Trademark Office, the Abstract of this application is provided for the purpose of satisfying the requirements of 37 C.F.R. §1.72 and the purpose stated in 37 C.F.R. §1.72(b) “to enable the United States Patent and Trademark Office and the public generally to determine quickly from a cursory inspection the nature and gist of the technical disclosure.” Therefore, the Abstract of this application is not intended to be used to construe the scope of the claims or to limit the scope of the subject matter that is disclosed herein. Moreover, any headings that may be employed herein are also not intended to be used to construe the scope of the claims or to limit the scope of the subject matter that is disclosed herein. Any use of the past tense to describe an example otherwise indicated as constructive or prophetic is not intended to reflect that the constructive or prophetic example has actually been carried out.

For any particular chemical compound disclosed herein, the general structure or name presented is also intended to encompasses all structural isomers, conformational isomers, and stereoisomers that may arise from a particular set of substituents, unless indicated otherwise. Thus, the reference to a general structure or name encompasses all enantiomers, diastereomers, and other optical isomers whether in enantiomeric or racemic forms, as well as mixtures of stereoisomers, as the context permits or requires.

The present disclosure is further illustrated by the following examples, which are not to be construed in any way as imposing limitations upon the scope of the claims. On the contrary, it is to be clearly understood that resort may be had to various other aspects, embodiments, modifications, and equivalents thereof which, after reading the description herein, may suggest themselves to one of ordinary skill in the art without departing from the spirit of the present disclosure.

### EXAMPLES

### Example 1

### High Throughput-Screening of Substrates or “Hit” Polymers that Support Cell Growth

Human pluripotent stem cells (hPSCs) include human embryonic stem cells (hESCs) and human induced pluripotent stem cells (hiPSCs), and the in vitro culture systems for the long term maintenance of hESCs and hiPSCs are remarkably similar. The screening reported here was conducted by using a well established hESCs line (BG 01) in an effort to identify polymer microspots that could be used for a range of hESC and hiPSC lines. A high throughput-based approach was employed to engineer new culture substrates that could be used to clonally expand human pluripotent stem cells in a chemically defined, xeno-free, feeder-free system.

To facilitate rapid synthesis and analysis of synthetic substrates, cell-compatible, biomaterial microarrays were manufactured.³³⁻³⁵ Polymer microarrays allow for rapid, nano-liter scale synthesis and analysis of libraries of polymeric surfaces on a standard glass microscope slide. Microarrays were prepared from 22 acrylate monomers with diversified hydrophobicity/hydrophilicity and crosslinking densities (FIG. 1a-b). The arrays were prepared by copolymerization between each of 16 “major” monomers (numbered 1-16) and each of 6 “minor” monomers (lettered A-F) at six different ratios [100:0, 90:10, 85:15, 80:20, 75:25, 70:30 (v/v)]. In this way, arrays with 496 [16+(16×5×6)] different combinations were created, comprised of the major monomer (70-100%) and minor monomer (0-30%). These monomer mixtures were robotically deposited in triplicate on a non-cell adhesive layer of poly(hydroxyl ethyl methacrylate) covered conventional glass slides (75 mm×25 mm), and then polymerized with a long-wave UV source.

Fluorescence-activated cell sorting (FACS) of transgenic hES cells were used to ensure that hES cells were both dissociated with each other and undifferentiated in the assays. High-throughput screening of biomaterials for clonal growth is provided as follows: First, transgenic Oct4-GFP hES cells were maintained on mEFs. Then, flow cytometry enabled the isolation of high purity undifferentiated hES cells from the completely dissociated coculture of hES cells and mEFs. Next, sorted cells were seeded onto polymer array. Finally, cellular response on polymer array was quantified by using laser scanner cytometry.

A transgenic green-fluorescent protein (GFP) reporter for Oct4 expression, a marker of pluripotent cells was knocked-in to the BG01 hES cell line and propagated under standard hES cell culture conditions utilizing mEFs.³⁶ Co-staining with primaries against GFP and Oct4 in BG01-Oct4-GFP hES cells were cultured on mEFs. Fluorescence of secondary antibody staining (488 nm for anti-GFP 1° antibody and 546 nm for anti-Oct4 1° antibody) indicated that all cells stained for GFP also stain for Oct4. At the fluorescent exposures used for these images, GFP fluorescence can not be detected at 488 nm or 546 nm unless it is stained by an anti-GFP antibodies. However, FACS can easily detect GFP expression without staining and GFP can be imaged at higher exposures. Oct4 reporter rapidly down-regulates upon differentiation and remains highly expressed when hES cells are in tightly packed colonies. hES cell differentiation was modulated by several factors in the media in 384 well plates for seven days and then GFP intensity was measured through immunostaining for GFP. Differentiation with BMP4 even in the presence of mEFs indicated a great knockdown of GFP intensity while GFP was rescued by increasing levels of FGF2.

GFP⁺ sorted hES single cells were seeded onto the polymer arrays and cultured with mEF-conditioned medium, since soluble growth factors secreted by mEFs help maintain the undifferentiated hES cell state.^{20, 21, 30} FACS analysis for hES and hiPS cells was provided. hES cells harboring a transgene with the human Oct4 promoter driving GFP expression were propagated under standard growth conditions with mEFs.³⁶ Cells were passaged, trypsinized and FACS sorted. GFP⁺ cells were utilized for all array experiments with hES cells. Differentiated GFP⁻ cells and mEFs were not included in the GFP⁺ gate. It was noted that most Oct4⁺ cells are also SSEA4⁺. In addition, SSEA4⁺ hiPS cells were utilized for all array experiments with hiPS cells.

It was shown that mEF-conditioned media can support propagation of hES cells when passed mechanically as clusters on matrigel but not when dissociated into single cells. Using standard protocols for generating mEF-conditioned media,²⁰ mEF-conditioned media were used for 3 days of culture. Phase contrast images indicates significant cell death and poor attachment when cultures were seeded as single cells, versus robust colony growth when seeded as clusters. Clusters differentiated on gelatin even in mEF-condition media when seeded on gelatin. In addition, a small molecule Rho-associated kinase (ROCK) inhibitor, Y-27632, was added to the media for the first 24 hrs of culture to reduce initial apoptosis of completely dissociated hES cells.³¹

Proteins can rapidly adsorb onto the surfaces of materials used for cell culture³⁷⁻⁴⁰. The surface properties of cell culture substrates can modulate both the amount and the conformation of adsorbed proteins, and thereby interact with cell surface receptors (e.g., integrins) to initiate signal transduction and alter cell behavior.⁴¹ To investigate the potential of different adsorbed proteins, fibronectin, laminin, bovine serum albumin (BSA), and fetal bovine serum (FBS) were separately adsorbed onto the microarrays from solution. In general, FBS was found to most effectively support the propagation of hES cells across the entire array, while fibronectin and laminin coatings led to more differentiation as indicated by down regulation of Oct4-GFP expression. Live-imaging of Oct4 expression in hES cells cultured on arrayed spots was provided with phase contrast and 488 fluorescence images of BG01-Oct4-GFP knocked-in hES cells cultured on polymer spots coated with different proteins (FBS, fibronectin, Laminin). Poor cell attachment was observed when arrays were coated with BSA. Therefore, FBS was used initially to coat the polymer array to screen for the suitable polymers (“hits”) that can support hES cells growth from single cells.

The FBS-coated arrays were seeded at low cell density (40 cells/mm² growth area of the array surface), to best model the ability of cells to grow in isolation. Two to ten non-contacting cells were observed on most polymer spots after 24 hrs cell culture. For instance, single Oct4-GFP⁺ hES cells were seeded on the polymer arrays. Cells were attached after one day of culture and provide a near clonal seeding density for each spot. For each polymer, the colony formation frequency was defined as the number of polymer spots on which Oct4⁺and SSEA-4⁺ hES cell colonies formed divided by the total number of replicate spots of the same kind of polymer on each array (n=3-18; see Methods). At various time points during cell culture, adherent cells were fixed and stained for cell nuclei and two pluripotent stem cell markers, SSEA-4 and Oct4. The cellular responses were quantified with laser scanning cytometry (LSC).⁴²

After 7 days of culture, a range of cellular responses was found on the polymer array upon subsequent culture in mEF-conditioned media: some polymers did not support either survival or growth of dissociated hES cells; some polymers supported the moderate growth of Oct4-differentiating cells; and potential “hit” polymers supported both robust growth and hES cell colony formation. For instance, the 16E-30% polymer did not support either attachment or survival of dissociated hES cells; the 6F-30% supported moderate growth but also differentiation of hES cells; and the 9 homopolymer (a “hit” polymer) supported robust growth of hES cells. These differences in cell response demonstrate that polymers can strongly modulate hES cell behavior between days 1 and 7 during colony growth from individual cells.

Phase-contrast images also indicated that cells can attach to the middle of the spot, as well as the edges. Colonies only in the middle of the spot also express pluripotency markers, SSEA4 and Nanog. Histogram of cells per polymer spot after 24 hrs of culture at a very low seeding density was also obtained, showing, for example, number of dissociated hES cells at day 1 on each polymer spot when the 15-A30% hit polymer array was seeded at a low density (3,000 cells per array).

To better understand the relationship between polymer chemical composition and clonal growth of hES cells, a map of colony formation frequency on the FBS-coated arrays against polymer monomeric composition was generated. In the map, major monomers were organized in order, from left to right, of increasing colony formation, while minor monomers were organized from bottom to top in order of increasing colony formation. Therefore, the region of the map corresponding to highest colony formation is the top right corner, while the region with the lowest is the bottom left corner. The frequency of colony formation on the primary polymer array was grouped into four categories 0-0.25, 0.25-0.50, 0.50-0.75, and 0.75-1.0 per polymeric spot. The map indicates that the homopolymer formed from monomer “5” poorly supported clonal growth of hES cells, while most other homopolymers effectively supported cell growth. Tertiary amine containing minor monomer “E” and oligo (ethylene glycol) containing minor monomer “A” negatively influenced colony formation frequency. While many polymers with a range of chemical moieties can support hES cell colony formation, several monomers (e.g., A and E) particularly seem to negatively impact cell growth.

### Example 2

### Qualify and Correlate Material Properties of Substrates to hES Cell Growth

All polymeric substrates in the library were characterized using high throughput techniques to quantify several materials properties: surface topographical roughness (in air, PBS, and culture medium after FBS adsorption), indentation elastic modulus (in air and fully hydrated in PBS), and surface wettability.⁴³ Surface roughness and elastic properties of bulk material substrata have been reported to affect the behavior of adult somatic cells^{44,45} and adult stem cells^{46,47}. Surface wettability—here quantified through measurements of the water contact angle (WCA)—indicates the hydrophobicity/hydrophilicity of polymer surface and has been correlated previously with protein adsorption and cell adhesion.⁴⁸

To develop quantitative relationships between the colony formation and material properties, the correlation of these properties with colony formation frequency was determined using linear and nonlinear regression that was plot in which polymer spots of distinct composition are clustered as a function of the property indicated on each horizontal axis; this representation avoids visual overweighting of properties which were not observed frequently across the polymer array. The data indicate that polymer surface roughness in air (root mean square, RMS˜0-25 nm), in PBS (RMS˜0-50 nm), and in culture medium after FBS adsorption (RMS˜0-110 nm) did not correlate strongly with colony formation frequency.

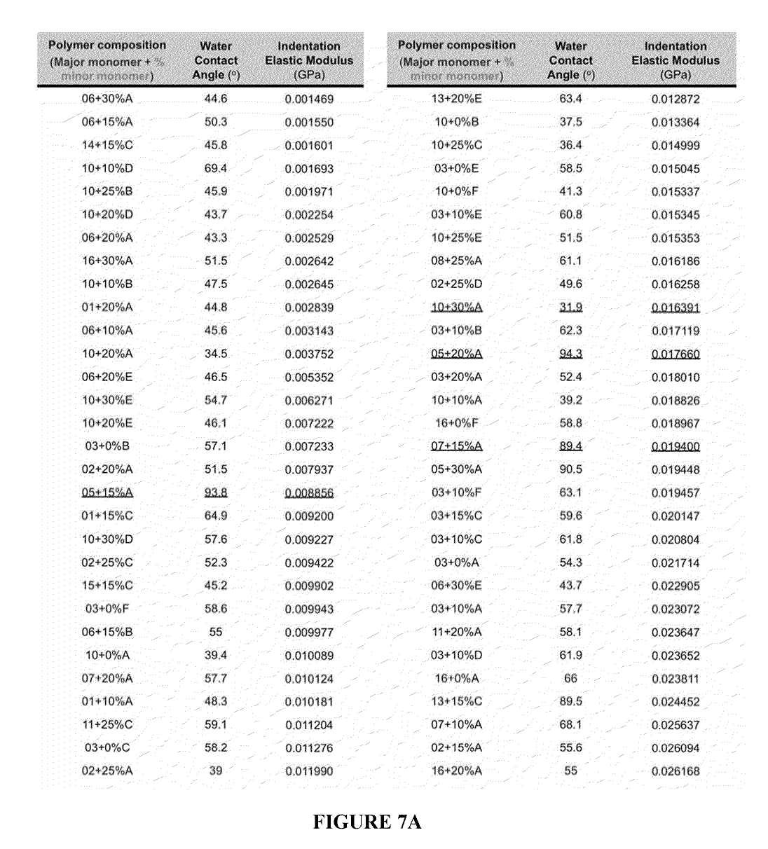

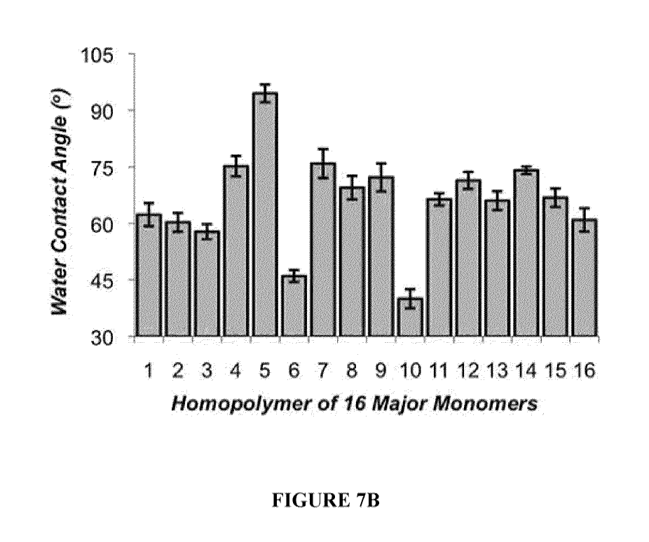

Variance in water contact angle were measured (FIGS. 7a-7b). Materials with similar reduced indentation elastic modulus could have very different WCAs. Standard error of measurement of WCAs was low for replicate samples (e.g., for WCA, <0.9-6.9%), as indicated by very consistent results in WCA measurements on 6 replicates of 16 homopolymers, whereas the standard error of measurement of roughness indicated a weak correlation of roughness with colony formation frequency.

However, a positive power-law correlation was observed between the indentation elastic modulus Eᵢ of hydrated polymers and colony formation frequency. It was also noted that polymers exhibiting a low indentation elastic modulus (i.e., high elastic compliance) also exhibited a low WCA. Many of these highly compliant polymers contain hydrophilic major monomer 10 and hydrophilic minor monomer A, which is consistent with the observation that the most compliant of these hydrated polymers also exhibited the greatest change in E, between the dry and fully hydrated states. This trend is consistent with previous studies of tissue cell adhesion and proliferation capacity on swellable polymers, wherein decreased elastic stiffness correlates directly with increased absorption of aqueous solvents.⁴⁹ Thus, the power-law correlation between Eᵢ and colony formation frequency, therefore, likely reflects the extent of polymer hydrophobicity/hydrophilicity in the cases where a hydrophilic polymer swells to create a compliant surface (Eᵢ<200 MPa) that poorly supports colony formation. Furthermore, it also demonstrated that, for the present array of hydrated polymers, colony formation is not strongly governed by polymer stiffness for Eᵢ of exceeding 0.2 GPa.

In contrast, a moderate wettability (WCA˜70°) is associated with optimal hES cell colony formation frequency. A contour projection of colony formation frequency as a function of both Eᵢ and WCA shows clearly that the optimum wettability (65°<WCA <80°) persists over a broad range of polymer stiffness, even for Eᵢ>200 MPa. Thus, together these data indicate that colony formation frequency of hES cells can be modulated most strongly by the WCA of these polymers, which is governed by multiple surface features including surface energy and topography, than by variation in the elastic moduli of these polymers over the range considered.

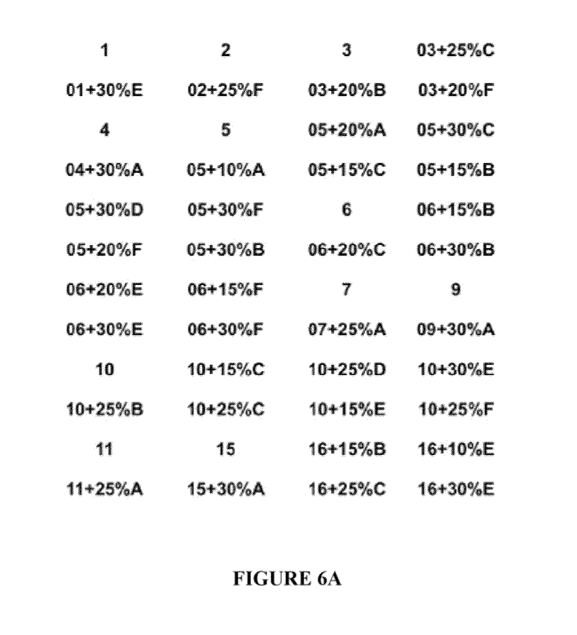

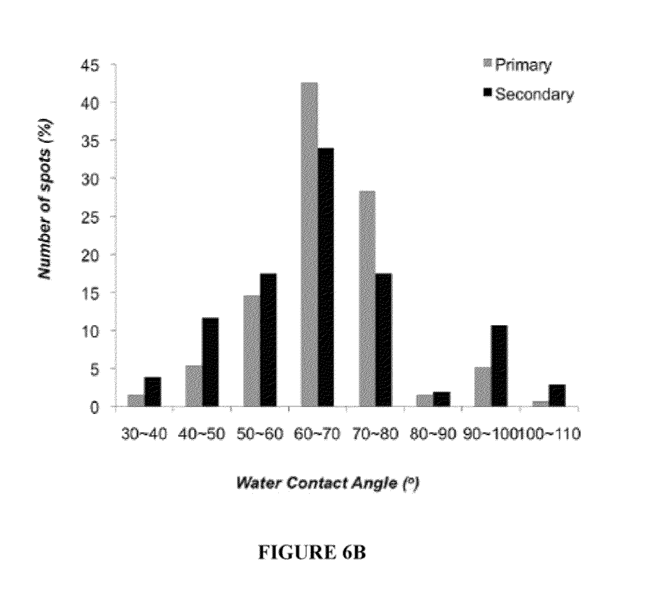

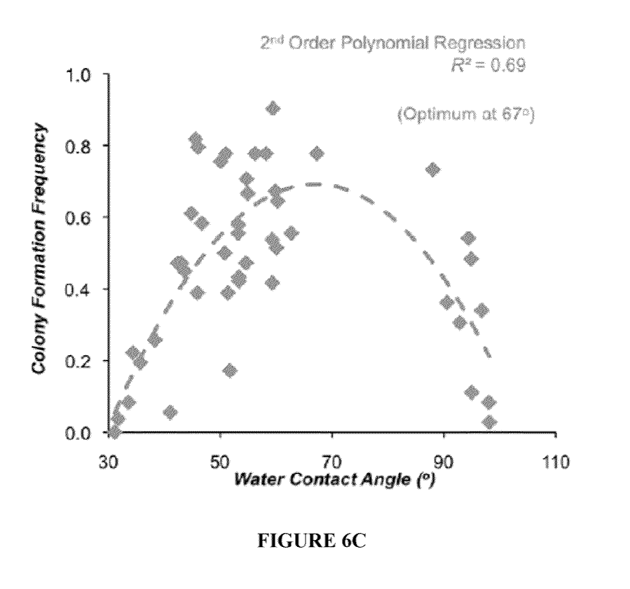

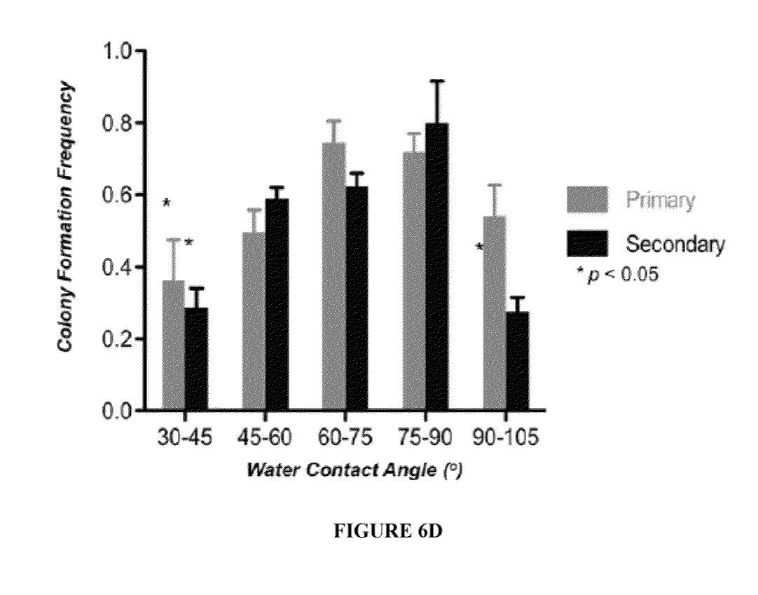

To further understand on how this combination of material properties, especially polymer wettability, modulates colony formation, 48 polymers were selected to generate a “secondary” polymer array with 36 replicates. This secondary array was designed to encompass a range of WCA similar to the range in the primary array (FIGS. 6a-6d), and the presence of twelve-fold more replicates per experiment significantly decreased experimental error. hES cell response to the secondary array also exhibited a wide range of behaviors as seen with the primary array.

In good agreement with the primary array data, a moderate wettability (WCA˜70°) again effectively supported optimal hES cell clonal growth (FIGS. 6c-6d). Surfaces of all polymers in the secondary array were analyzed using time-of-flight secondary ion mass spectrometry (ToF-SIMS) in a high throughput manner to provide molecular information of the topmost layers (˜10 Å) of each polymer surface.^{50,51} ToF-SIMS spectra from two homopolymers generated from similar monomers 1 and 16 (FIGS. 3a-3b) were substantially different, suggesting that the polymer surface chemistry cannot be necessarily predicted from the monomer composition alone.

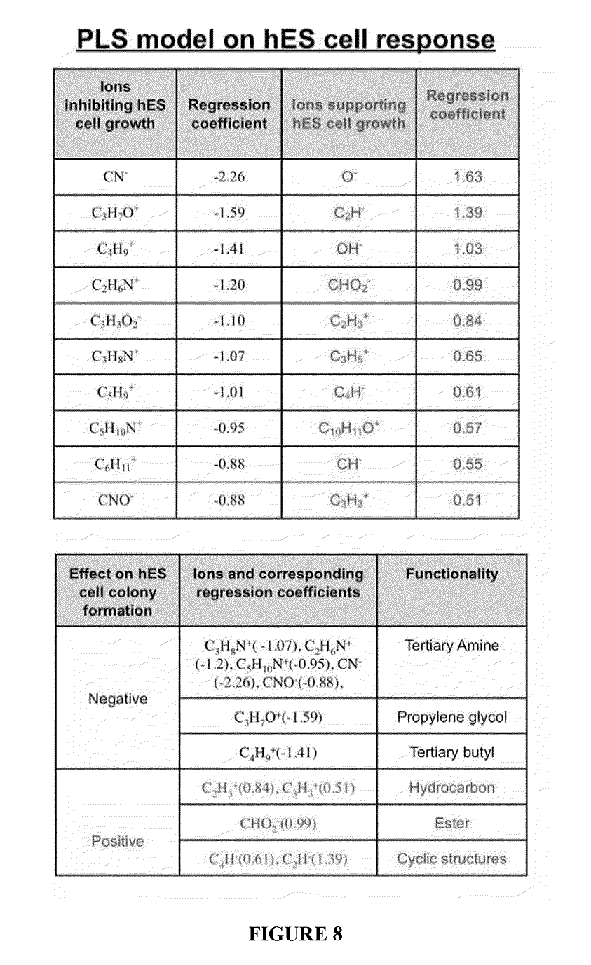

Using a chemometric technique (partial least squares (PLS) regression on ToF-SIMS spectra),^{43,51} surface chemistry contained in the spectra was correlated to the colony formation frequency observed on each polymer in the secondary array. Good agreement between measured colony formation frequency and that predicted from the ToF-SIMS spectra was found (R²=0.78). Each secondary ToF-SIMS ion associated with functionalities in the polymer structures could be listed with its regression coefficient, “α”, a quantitative measure of its contribution to colony formation frequency (FIG. 8). The tertiary amine moiety (characteristic ions C₃H₈N⁺, C₂H₆N⁺, CN⁻) and tertiary butyl moiety (C₄H₉⁺) was identified by the PLS analysis to be correlated most strongly with a low colony formation frequency, while hydrocarbon ions (C₂H₃⁺, C₃H₃⁺), oxygen containing ions (CHO₂⁻, C₃H₃O⁺, C₂H₃O⁺) from esters and ions from cyclic structures (C₆H⁻, C₄H⁻, C₂H⁻) had the largest effect on promoting colony formation. The oxygen containing ions and hydrocarbon ions can be attributed to the acrylate groups in each monomer which form the backbone chain and the pendant ester groups after polymerization. Monomers with di- and tri-acrylates, which contain the most acrylate groups in our library, indeed showed the highest colony formation frequencies.

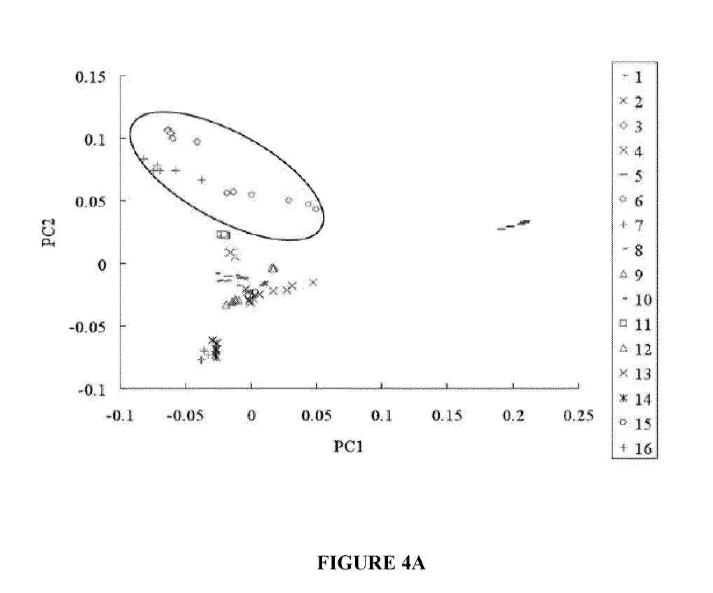

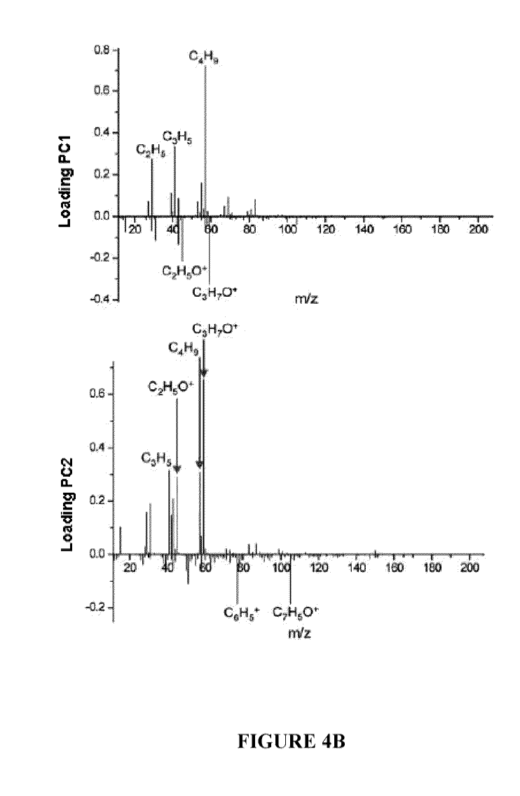

The refined quantitative relationships among surface chemical structure and hES cell clonal growth generated from the secondary array provides an integrated view of all the cell responses seen in the dataset and may be further used to predict the performance of new hES/hiPS cell culture materials. For example, the relationship between surface chemistry and colony formation frequency established using the ToF-SIMS from the secondary array consistent with hES cell behavior seen on the primary array. On the primary array, the pendent functional groups in mono-acrylates (4, 5, 7, 10) have sizeable effects on colony formation. For instance, the presence of tertiary butyl, a large non-polar functional group (α<0), in the major monomer 5 resulted in low colony formation. For most di- and tri-acrylates major monomers (1, 2, 8, 9, 11, 12, 13, 14, and 15) in the primary array, high acrylate content supported robust clonal formation as expected from the large positive α. The exceptions (3, 6, 16) can be attributed to the presence of a long chain of propylene glycol/ethylene glycol (for glycols, α<0). Although the ethylene glycol moiety can be found in the monomer chemistry of additional di-acrylates major monomers such as 1, 2, 9, 11, ToF-SIMS analysis indicated much higher propylene/ethylene glycol content present at the surface of homopolymers 3, 6, and 16 compared to 1, 2, 9, and 11 (FIGS. 4a-4b).

Further, the PLS model based on the secondary array data was used to predict hES cell colony formation of all 16 homopolymers in the primary array based entirely on their ToF-SIMS spectra. In this analysis, reasonable agreement between predicted and measured hES cells colony formation frequency (R²=0.7) of the 16 homopolymers is observed. This demonstrates that the model can be used to quantitatively predict hES cells clonal growth on a variety of acrylate polymers outside of the training set of the model. Lastly, polymers with high-acrylate content generally have a moderate WCA which is consistent with the colony formation peak.⁵² The biological performances of polymer substrates depend on the combined effects of chemical moieties present on their surfaces, and this analysis provides insight into the common characteristics of polymers for optimal hES cell colony formation.

### Example 3

### Validation of Cellular Performance with “Hit” Polymers

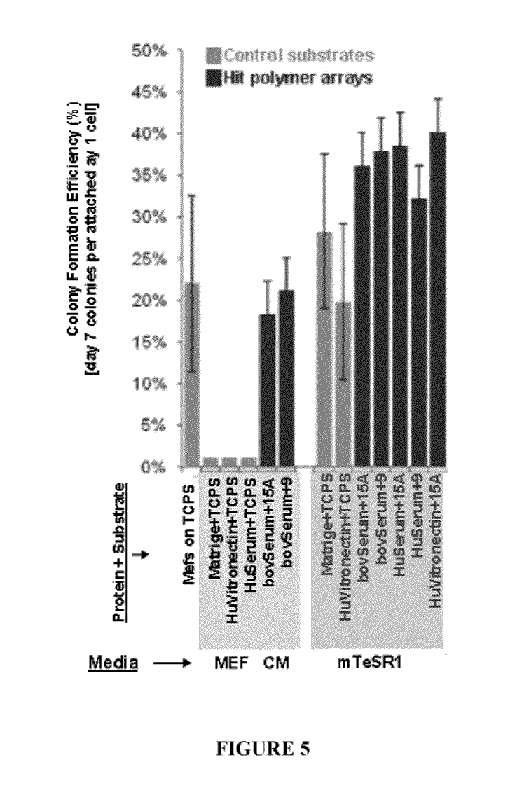

Since polymers with a moderate WCA generated from multiple acrylate groups performed best in these experiments, the homopolymer of monomer 9, a di-acrylate with phenyl groups, and the copolymer with 15-30% monomer A, a tri-acrylate, were chosen to further validate cellular performance with a collection of biological assays. “hit” arrays were fabricated where the entire polymer array is composed of one “hit” polymer (i.e., 9 or 15A-30%). The colony formation efficiency of mEFs and “hit” polymer spots was quantified based on the ratio of hES cells colonies formed on day 7 per attached hES cell on day 1 (FIG. 5). About 20-25% of attached hES cells on day 1 created GFP⁺, SSEA4⁺ undifferentiated hES cell colonies after seven days of culture on either the mEFs substrate or on the hit polymers.

In contrast, cells on vitronectin and matrigel coated tissue culture polystyrene (TCPS) exhibited predominately differentiated growth, had lower Oct4 expression, and did not form typical hES cell colonies with distinct borders. Further, nearly all (>95%) spots on hit arrays can support the expansion of Oct4⁺, SSEA4⁺, Nanog⁺, and Tra1-60⁺ cells after 7 day culture from completely dissociated hES single cells, and similar behavior was seen with other pluripotent cell lines: an hiPS cell line, and a non-transgenic hES cell line. It was shown that human serum-coated hit polymers 9 and 15-A30% supported SSEA4⁺ colony formation of C1 hiPS cell line, and SSEA4⁺, Nanog⁺ colony formation of mechanically passaged WI33 hES cell line.

The hit arrays were further evaluated for their capacity to maintain pluripotency of hES cells after prolonged cell culture. In these studies, hiPS cells were immunostained against SSEA-4, and then the SSEA4+ FACS sorted cell population was used. Immunostaining of dissociated hES cells propagated on hit FBS-coated “15A-30%” polymer for 7 days against Nanog (green) and Tra-1-60 (red), and on FBS-coated hit “9” polymer for 7 days against Oct4 and SSEA-4 was performed. Phase contrast images dissociated hES cells cultured on hit polymer “9” array for more than 2 months (>10 passages), and these cells were then moved onto mEFs. Cell remained positive for pluripotent markers: Tra-1-60 and Nanog, were seen. Karyotypic analysis of hES cells propagated on hit “9” polymer array for more than 2 months (>10 passages) was also performed. It was shown that a normal 46XY karyotype was maintained on the hit array. Gene expression analyses via RT-PCR of various differentiation markers for the three germ layers generated through embryoid body (EB) in vitro differentiation, and teratoma formation in immunodeficiency mice by cells cultured on “15A-30%” hit arrays were also performed. H&E staining was performed on the teratoma. Resulting teratoma contained tissues representing all three germ layers: ectoderm, epidermal and neural tissue (rosette); mesoderm, bone and cartilage; and endoderm, respiratory epithelium and intestinal-like epithelium.

Therefore, after more than 2 months of culture (>10 passages) on the polymer “hit” arrays, cells were found to maintain an undifferentiated state by expressing hES cell markers including Oct4, Nanog, Tra1-60 and SSEA4. In addition, hES cell colonies appeared when they transferred to mEFs after >10 passages on the “hit” polymer array, immunostaining of dissociated hES cells propagated on FBS-coated polymer hit polymers for 7 days after lone term culture showed strong expression of the typical hES pluripotency cell markers: Oct4(GFP), SSEA4, Nanog, and Tra1-60. Cells also remained positive for pluripotent markers: Oct4, Nanog, and Tra1-60, and Sox2 when cultured on human serum coated hit arrays for 5 passages and then moved to mEFs substrates. Clonal efficiency of cells after long-term culture remained ˜20%. Additionally, a normal karyotype showed the capacity of both “hit” polymers (9 and 15A-30%) to maintain the genetic integrity of hES cells after a long-term culture. Gene expression results confirmed robust differentiation of these hES cells into all three germ lineages after 13 days of embryoid body cultivation, and derivatives of all three embryonic germ layers were seen in teratoma assays. These results demonstrate that hES cells cultured on the polymeric hits maintain their full pluripotent potential.

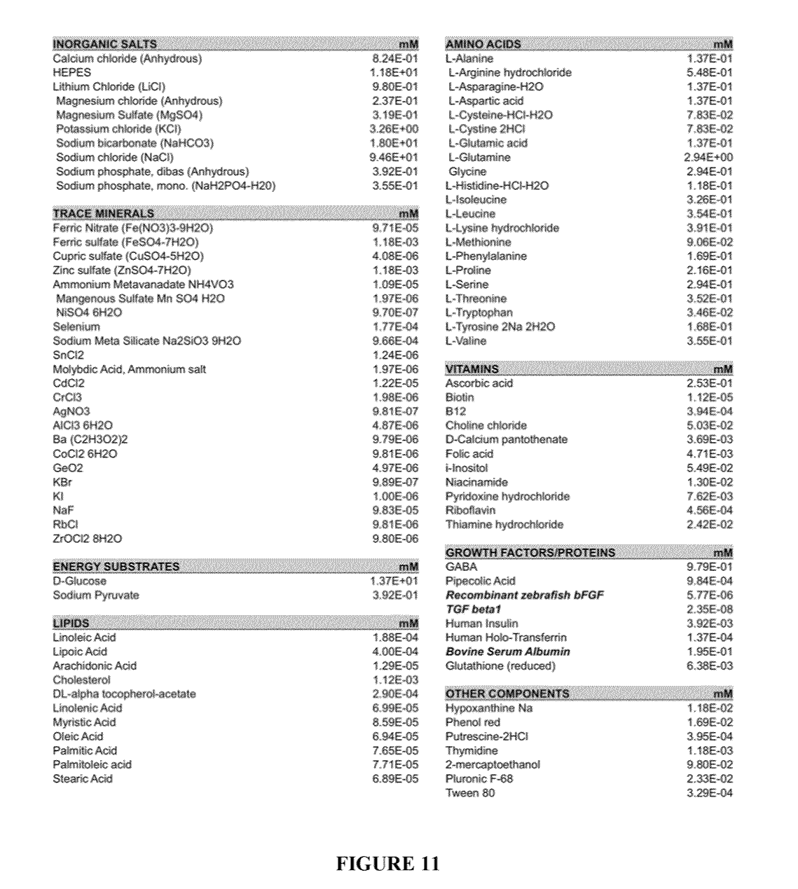

To develop a more clinically relevant, defined culture system for hES cells, long-term culture was conducted on human serum coated (HS) “hit” polymer arrays in mTeSR1 medium, a completely chemically defined media (FIG. 11). HS-coated “hit” polymer arrays supported the expansion of dissociated hES cells in a similar manner to arrays coated with FBS. Further, the HS-coated hit arrays could support long-term culture for more than 1 month (>5 passages), with robust expression of hES cell markers including Oct4 and SSEA4. Lastly, the HS-coated hit polymers could support the undifferentiated growth of hiPS and other hES cell lines.

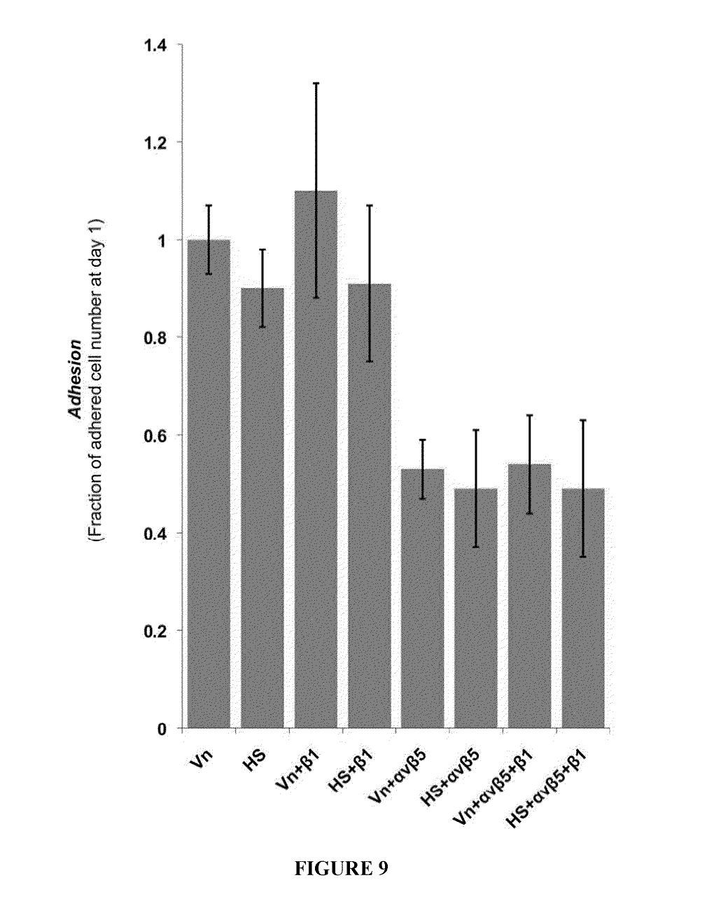

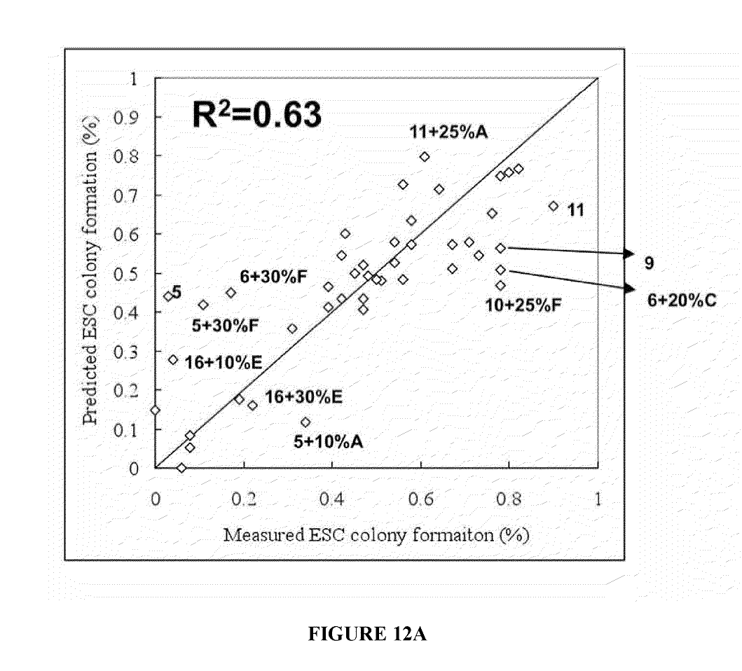

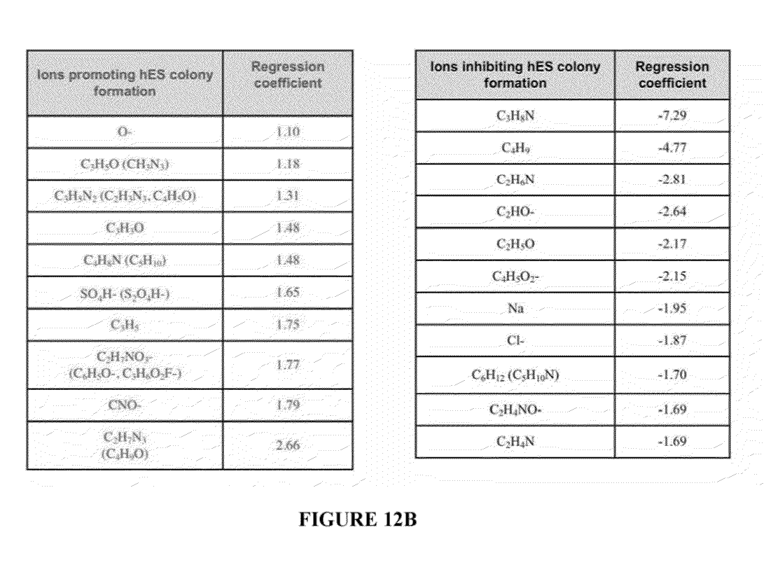

To investigate the potential pathways through which human serum may be important for colony formation, integrin blocking experiments were performed on the hit polymers (FIG. 9) for several highly expressed integrins^{53,54}. Blocking only integrin αᵥβ₅, the vitronectin-binding cell surface receptor, showed the most significant decrease in day 1 adhesion, while blocking an important matrigel binding cell surface receptor⁵⁴, integrin β₁, had no effect. Vitronectin is also abundantly present in serum⁵⁵, and we tested its capacity to support colony formation when coated on the hit polymers. Similar levels of hES cell adhesion at day 1 were observed on the FBS- or HS-coated “hit” polymers and the vitronectin-coated “hit” polymers (FIG. 9). The colony formation efficiency of dissociated hES cells at day 7 on FBS/HS coated “hit” polymer arrays was identical to the efficiency on vitronectin-coated “hit” polymers. The histogram of the cell number on the polymer spots at day 1, indicated that the majority of colonies formed at day 7 are expanded from a single cell. Although vitronectin-coated TCPS was recently reported to support the expansion of hES cells²³, these surfaces were not demonstrated to support hES cell clonal growth, and significant differentiation was observed during clonal growth. These results indicate that the surface chemistry of hit substrates interact with vitronectin, which engages with proper hES cell surface receptors (integrin αᵥβ₅) to support the clonal growth of human pluripotent cells. Surface chemical analysis on vitronectin-coated polymer arrays were also performed (FIGS. 12A-12B) to gain insight on whether vitronectin adsorption alone could predict biological performance of our polymers. The resulting ToF-SIMS spectra on secondary arrays was correlated with hES colony formation using PLS-modeling, and a considerably poorer correlation (R²=0.63; FIG. 12A) was found when using vitronectin-coated polymer array spectra in the model than using bare polymer array spectra (R²=0.78).

In summary, the biological activities of polymeric substrates can be controlled by surface properties, which in turn are determined by chemical moieties present on the polymer surface. However, it can be difficult to quantitatively predict the presence of certain chemical functional groups at the polymer surface from the monomer composition alone, as well as the effects of surface chemistry on biological performance⁵⁶. Here, high throughput materials synthesis and analysis were utilized to rapidly establish quantitative relationships between surface chemical structures and hES cell clonal growth. The structure-function relationships described herein reveal that acrylate-based polymers with a moderate wettability (WCA˜70°) optimally support clonal growth. Surface roughness and indentation elastic modulus are not strongly correlated with clonal growth, within the ranges evaluated here (except to the extent that modulus is correlated with wettability). Integrin αᵥβ₅ engagement with vitronectin was further identified as important for clonal growth on the array. Top-performing substrates were characterized and provide substrates superior to existing hES cells culture substrates including vitronectin-coated TCPS, matrigel, and mEFs. The hit substrates supported the clonal growth of karyotypically normal hES cells at unprecedented high levels for “chemically defined, xeno-free, feeder-free” hES cell culture substrates. The combination of human vitronectin-coated “hit” polymers and mTeSR1 media provide an attractive platform to develop a fully chemically defined, xeno-free, feeder-free culture system, as the only animal component, BSA from the mTeSR1 medium, can be replaced by human serum albumin. Together, these advances may permit the facile growth of hES/hiPS cells from fully dissociated single cells, thereby enabling more straightforward genetic manipulation.

The following provides methods for the studies discussed herewith.

Combinatorial Array Preparation: Polymers were printed in a humid Ar-atmosphere on epoxy monolayer-coated glass slides (Xenopore XENOSLIDE E, Hawthorne, N.J.) which were first dip-coated in 4 vol.-% poly(hydroxyethyl methacrylate), using modifications of robotic fluid-handling technology as described previously^{33,34}. Spots were polymerized via 10 s (second) exposure to long wave UV (365 nm), and dried at <50 mtorr (1 torr=133.32 Pa) for at least 7 days. The chips are sterilized by UV for 30 min for each side, and then washed with PBS twice for 15 min to remove the residue monomer or solvent. Finally, the chips were coated with various proteins: 20% FBS (v/v, Hyclone) at room temperature for 15 min, BSA (1 mg/mL, Sigma) at room temperature for 1 hr, laminin (4 μg/mL, Sigma) at 37° C. for 2 hr, human fibronectin (25 μg/mL, Sigma) or human vitronectin (Invitrogen; 1-3 μg/mL in DMEM) at 37° C. for 1 hr, or 20% human serum (v/v, Sigma) at room temperature for 15 min. These surfaces were then washed with cell culture medium before cell seeding.

Surface Roughness Measurements: Surface roughness measurements were taken using a Digital Instruments Dimensions 3000A AFM instrument in tapping mode. The automated acquisition of height and phase measurements for all polymer spots on the primary array was achieved by calculating the coordinates of each polymer spot and inputting these values into the programmed move feature of Nanoscope 5.31R1 software. Measurements were taken in both air and fluid. In air, silicon tips with a resonant frequency of approximately 300 kHz and a force constant of 40 N/m were used (Tap300Al, Budget Sensors). In fluid, silicon nitride tips with a resonant frequency of approximately 7 kHz and a force constant of 0.58 N/m were used (DNP-S, Veeco). Tapping mode was achieved using Z-modulation. Solutions used were either Milli-Q water or DMEM (GIBCO) containing 25% FBS (Hyclone) and supplemented with non-essential amino acids and L-Glutamine. Samples were incubated with the solution for 24 hours before AFM measurements were conducted and were kept in solution until all polymer spots were sampled. 5 μm regions of the polymer were taken and the root mean square (RMS) roughness was measured across this region. Image processing was conducted using SPIP V3.3.6.0 software.

Water Contact Angle Measurements: Measurements were the sessile drop type and performed using ultra pure water on a Kruss DSA 100 apparatus fitted with a piezo-doser head. The piezo-doser allowed small ultra pure water droplets (110 pL) to be deposited onto the polymer spots. Sample positions and data acquisition were automated, with droplet side profiles being recorded (a dual camera system was used, one to record a spot's side profile and the other to record a bird's eye view to ensure that the water droplet was deposited at the centre of each spot) for data analysis. WCA calculations were performed using a circle segment function as required for small water droplets.

Elastic Modulus Measurements: Instrumented indentation was conducted on the primary array, comprising polymer spots with an average diameter of 300 μm, height of ˜15 μm with center-to-center distance of 740 μm, using a pendulum-based instrumented nanoindenter (NanoTest, Micro Materials Ltd.). The array glass slide was mounted on an aluminum support with a thin layer of cyanoacrylate. Experiments were conducted in ambient air, as well as upon full immersion and hydration of the array in PBS at room temperature. Hydrated arrays were immersed in PBS for at least 12 hours prior to indentation to achieve equilibrium hydration, and maintained in this hydrated state throughout the experiment using a modified platform for in situ liquid experiments.⁵⁷ Samples were indented with a spherical ruby indenter of radius R=500 μm, (n=3 locations for each polymer spot), with loading and unloading rates of 0.5 mN/s, dwell of 10 s and a maximum load of 3 mN or a maximum depth of 600 nm, depending on which limit was attained first. This condition was chosen such that the average strains imposed on the polymer spots (estimated as a/R, where a is the contact radius) was less than 5% on all samples and the ratio of maximum indentation depth hₘₐₓ to polymer spot thickness t was maintained less than 4% on all samples; this low hₘₐₓ/t minimized contributions from the stiff glass support to the measured elastic response, and data were not corrected for finite thickness because the hydrated thickness was not measurable with high accuracy for all polymers. Loading rates were chosen such that the reduced elastic modulus inferred from indentation, Eᵣ could be calculated from the initial unloading slope through Oliver and Pharr method.^{58,59} Indentation elastic modulus presented in the manuscript was calculated from the measured reduced elastic modulus, assuming a Poisson's ratio v of 0.49 for all polymers in the array.

Time of flight secondary ion mass spectroscopy (ToF-SIMS or simply, SIMS): A secondary ion mass spectrometer (ION-TOF, IV, UK) was operated using a Bi₃⁺ primary ion source operated at 25 kV and in “bunched mode”. A 1 pA primary ion beam was rastered at an area of 100×100 μm. Secondary ions were collected from the same area of each polymer spot on the microarray over 10-second acquisition time. Ion masses were determined using a high-resolution Time-of-Flight analyser allowing accurate mass assignment. The typical mass resolution (at m/z 41) was just over 6000.

Data Regression and Visualization: Linear and nonlinear least-squares regression was performed with Excel (Microsoft). Contour and 3D plots were generated in MATLAB 4 (Mathworks) using the v4 griddata method of data interpolation. The method defines a smooth surface fit to the data.

Cell Culture: hES cell lines BG01 (National Institutes of Health [NIH] code: BG01; BresaGen, Inc., Athens, Ga.) and WIBR33 (Whitehead Institute) were maintained on mitomycin C (MMC)-inactivated mouse embryonic fibroblast feeder (mEFs) layers in hES cell medium (Dulbecco's modified Eagle's medium DMEM/F12 [Invitrogen] supplemented with 15% FBS [Hyclone], 5% KnockOut Serum Replacement [Invitrogen], 1 mM glutamine [Invitrogen], 1% nonessential amino acids [Invitrogen], 0.1 mM β-mercaptoethanol [Sigma], penicillin/streptomycin [Invitrogen], and 4 ng/ml FGF2 [R&D Systems]). Cultures were grown at 37° C. in 5% O₂ and were passaged every 5 to 7 days either manually or enzymatically with collagenase type IV (Invitrogen; 1 mg/ml for 10 min).

hES BG01-Oct4-GFP cells were made by introducing a Oct4-GFP-puro construct into hES cells.³⁶ In this construct, the GFP reporter gene is expressed from the human Oct4 promoter that is active when cells are in an undifferentiated state. Upon differentiation, the Oct4 promoter is gradually inactivated and therefore the GFP reporter is down-regulated. At the time of this study, this BG01-Oct4-GFP line had been cultured over 30-95 passages with 46XY normal karyotype. This line expresses all pluripotent stem cell markers and forms teratomas after being grafted into severe combined immunodeficient mice (SCID).

hiPS C1 cells were derived through lentiviral infection of Oct4, Sox2, and Klf4 and cultured in hES cell media on mEFs as described previously.⁶⁰ At the time of this study, this line had been cultured for 5-15 passages on mEFs.

For EB-induced differentiation, hES cell colonies were harvested with 1 mg/ml collagenase type IV (Invitrogen), separated from the mEF cells by gravity, gently triturated, and cultured for 13 days in nonadherent suspension culture dishes (Corning) in DMEM supplemented with 15% FBS.

Flow Cytometry: For FACS sorting, hES or hiPS cell lines were cultured in 10 μM Rho Kinase (ROCK) inhibitor (Calbiochem; Y-27632) for 24 hr in standard mEF conditions prior to sorting. Cells were harvested enzymatically with collagenase type IV (Invitrogen; 1 mg/ml), and then with 0.05% trypsin/ethylenediaminetetraacetic acid (EDTA) solution (Invitrogen) for 5 minutes at 37° C. hiPS cells were labeled with immunostained using SSEA4 (mouse monoclonal, Developmental Studies Hybridoma Bank; 1:10 supernatant dilution in mTeSR1 media for 10-15 min at 4° C.) and Molecular Probes ALEXAFLUOR® 647 dye-conjugated secondary antibodies (Invitrogen; 1:50 for 10 min at 4° C.). Cells were collected in media with ROCK inhibitor and sorted on a FACSAria Flow Cytometer (Becton Dickinson, San Jose, Calif.). Cells were subsequently plated on various surfaces in medium supplemented with ROCK inhibitor for the first 24 hr. For the human serum coated hit arrays, culturing occurred in mTeSR1 media (Stemcell Technologies). For efficiency experiments on TCPS, single cells were sorted individually (1 cell/well) directly into each well of a 96 well plate (Corning) coated with human vitronectin (Invitrogen; 1-3 μg/mL in DMEM), FBS (20% in DMEM), 20% human serum (v/v, Sigma), or matrigel (Invitrogen; using supplier's thin gel method).

Karyotype Analysis: Chromosomal studies were performed by Cell Line Genetics (Madison, Wis.) using standard protocols for high-resolution G-banding.

Teratoma formation and analysis: hES Cells were collected by collagenase treatment (1 mg/ml for 10 min) and separated from feeder cells by subsequent washes with medium and sedimentation by gravity. hES cell aggregates were collected by centrifugation and resuspended in 250 μl of PBS. hES cells were injected subcutaneously in the back of SCID mice (Taconic). Tumors generally developed within 4-8 weeks and animals were sacrificed before tumor size exceeded 1.5 cm in diameter. Teratomas were isolated after sacrificing the mice and fixed in formalin. After sectioning, teratomas were diagnosed based on hematoxylin and eosin (H&E) staining.

Immunocytochemistry: Cells were fixed in 4% paraformaldehyde in PBS and immunostained according to standard protocols using the following primary antibodies: SSEA4 (mouse monoclonal, Developmental Studies Hybridoma Bank); Tra 1-60, (mouse monoclonal, Chemicon International); hSOX2 (goat polyclonal, R&D Systems); Oct-3/4 (mouse monoclonal, Santa Cruz Biotechnology); hNANOG (goat polyclonal R&D Systems); appropriate Molecular Probes Alexa Fluor® dye conjugated secondary antibodies (Invitrogen) were used. When necessary, cells were permeabilized with 1% Triton X-100 in PBS for 10 mins, and then stained. The chips were washed with PBS and water to remove the salts, and air dried. The chips were imaged with iCys laser scanning cytometry.

Multivariate analysis: Principal component analysis (PCA) and partial least squares (PLS) regression were carried out using the Eigenvector PLS_Toolbox 3.5. The SIMPLS algorithm was used for the PLS analysis. A “leave one out” cross validation method was used for the PLS analysis. Both ToF-SIMS and hES cell data were mean-centered before analysis. The Root Mean Square Error of Prediction (RMSPE) was calculated to quantify how well each model predicted the training set or test set polymers. The individual peak intensity was normalized to the total secondary ion count to remove the effect of primary ion beam fluctuation. The positive and negative ion intensity data was arranged into one concatenated data matrix. 181 positive and 43 negative ions were selected from a group of polymers from the array containing all 22 monomers to form the peak lists. The PLS model constructed from the training polymer samples produced a set of regression coefficients for each secondary ion. These regression coefficients were used to predict the hES cell colony formation on the test samples using their SIMS spectra. Due to variations in ion intensity, predicted frequencies were normalized.

Efficiency measurements: In FIG. 5, cell numbers and colony numbers were calculated as follows. Cell numbers on mEFs and matrigel on day 1 were quantified per scanned area; the cell number on “hit” polymer spots is quantified per “hit” array (1728 replicates) with a seeding density 3,000 cells/array (1.6 cells/mm² of array growth area). Colony numbers on mEFs and matrigel on day 7 were quantified per scanned area; the colony number of “hit” polymer spots was quantified per “hit” array (1728 replicates). On TCPS, cell numbers were measured by counting the number of wells on day 1 with cells after single cell sorting into individual wells of a 96 well plate. Colony numbers on TCPS were counted by staining wells after 7 days. For vitronectin-coated, matrigel-coated, and HS-coated TCPS in MEF-CM, no GFP+ colonies were observed: in about 30% of cases we observed only differentiated cell growth.

### Example 4

### UV/Ozone-Patterned Substrates for Human Cell Culture

Patterned substrates are heterogeneous culture substrates where cell adhesive regions separated by cell repulsive regions. The patterned substrates could provide ideal microenvironments for mammalian cell culture and manipulation. Patterned substrates can be prepared by a variety of techniques. One example is photolithography: using short wavelength UV treat virgin polystyrene (PS) in a spatially defined manner to create cell adhesive islands from cell repulsive substrates. Some examples were given here based on the results from human pluripotent stem cells. However, the usage of the substrates can be extended to other mammalian cell types. Some potential examples include hepatocytes, neural progenitors, and hematopoietic stem cells. In addition to the surface geometry, surface chemistry play a role. It is expected that different mammalian cell types may require different surface chemistries.

The following studies demonstrate UV/ozone-patterned substrate, e.g., polystyrene, for human cell growth.

Ultraviolet (UV) light-treated polystyrene share the same defining surface chemical features as the hit polymers. The UV/Ozone unit (Bioforce Nanoscience Inc., USA) was utilized to generate high intensity UV light, principally at 184.9 and 253.7 nm wavelengths, which excite molecular oxygen to form atomic oxygen and ozone. In this study untreated polystyrene (Corning) or ultralow attachment surface (Corning) was oxidized at a distance of around 4 cm from the UV lamp and results were reported for exposure times under atmospheric conditions after preheating the UV lamp for 30 min. Surfaces were subsequently coated with 20% human serum (v/v, Sigma) for 1 hr at room temperature.

Chemically heterogeneous surfaces (patterned surfaces) were obtained by a simple masking technique. The custom made stainless steel photomask was placed on the surface of the untreated polystyrene dish or ultralow attachment dish and treated in the unit as above. hES BG01-Oct4-GFP cell line was cultured in 10 μM Rho Kinase (ROCK) inhibitor (Calbiochem; Y-27632) for 24 hr in standard mEF conditions^{61,63} prior to sorting. Cells were harvested enzymatically with collagenase type IV (Invitrogen; 1 mg/ml), and then with 0.05% trypsin/ethylenediaminetetraacetic acid (EDTA) solution (Invitrogen) for 5 minutes at 37° C. Cells were collected in media with ROCK inhibitor and sorted on a FACSAria Flow Cytometer (Becton Dickinson, San Jose, Calif.). Cells were subsequently plated on various surfaces mTeSR1 media (Stemcell Technologies) supplemented with ROCK inhibitor for the first 24 hr. Culturing occurred in mTeSR1 media (Stemcell Technologies). Pluripotent colonies were assayed on day 7 by one of two methods: image analysis from taking twenty 100× phase contrast pictures and manual counting under a brightfield microsope.

hES Clonal growth (% of colonies formed on day 5 per cell seeded) on bacterial grade polystyrene that has been treated with UV was measured for various times. Optimal treatment occurred between 5-30 for this UV wavelength and power.

A secondary ion mass spectrometer (ION-TOF, IV, UK) was operated using a Bi₃⁺ primary ion source operated at 25 kV and in “bunched mode”. A 1 pA primary ion beam was rastered at an area of 100×100 μm. Secondary ions were collected from the same area of each polymer spot on the microarray over 10-second acquisition time. Ion masses were determined using a high-resolution Time-of-Flight analyser allowing accurate mass assignment. The typical mass resolution (at m/z 41) was just over 6000. Surface chemical analysis using time of flight secondary ion mass spectroscopy (TOF-SIMS) on conventional polystyrene surfaces versus UV-treated polystyrene was provided.

Partial least squares (PLS) regression were carried out using the Eigenvector PLS_Toolbox 3.5. The SIMPLS algorithm was used for the PLS analysis. A “leave one out” cross validation method was used for the PLS analysis. Both ToF-SIMS and hES cell data were mean-centered before analysis. The Root Mean Square Error of Prediction (RMSPE) was calculated to quantify how well each model predicted the training set or test set polymers. The individual peak intensity was normalized to the total secondary ion count to remove the effect of primary ion beam fluctuation. The positive and negative ion intensity data was arranged into one concatenated data matrix. Several positive and negative ions were selected from the spectra to form the peak lists. The PLS model constructed from the training polystyrene samples produced a set of regression coefficients for each secondary ion. These regression coefficients were used to predict the hES cell colony formation on the test samples using their SIMS spectra. Due to variations in ion intensity, predicted frequencies were normalized.

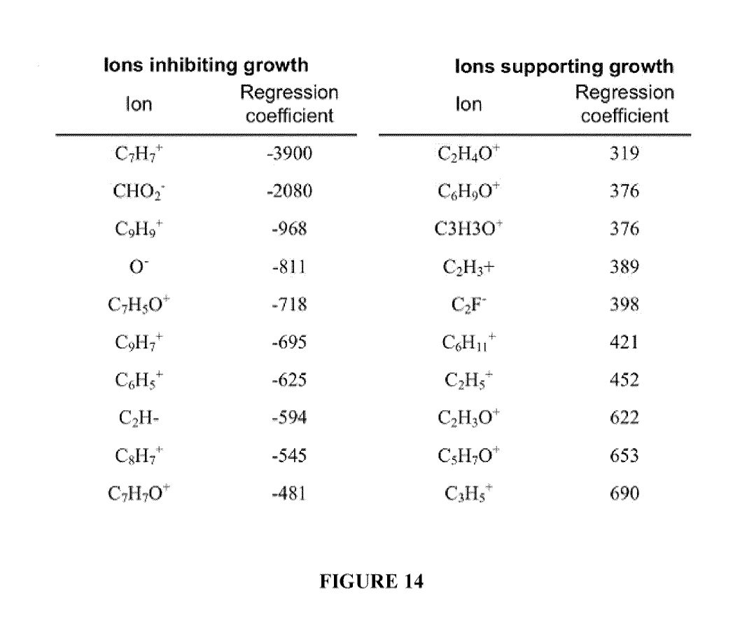

Using PLS-analysis on the TOF-SIMS data, this study provided characteristic ions supporting or inhibiting clonal growth on the UV/ozone treated polystyrene (FIG. 14). The PLS analysis of the ToF-SIMS spectra of the secondary polymer array revealed that hydrocarbon ions (C₂H₃⁺, C₃H₃⁺), and oxygen containing ions (CHO₂⁻, C₃H₃O⁺, C₂H₃O⁺) from esters had the largest effect on promoting colony formation of hESCs. PLS analysis of the ToF-SIMS spectra of UV/ozone treated polystyrene showed a similar trend. Hydrocarbon ions (C₂H₃⁺, C₃H₅⁺), and oxygen containing ions (C₃H₃O⁺, C₂H₃O⁺) from esters had the largest effect on promoting colony formation. It confirmed that UV/ozone treatment of PS surface could generate chemical ion signature similar to “hit” polymers. Predictions based on the ion signature of the UV/ozone treated surface and the experimentally measure clonal growth observed.

UV-treatments with mask can create patterns of adhesive/repulsive surfaces. Virgin polystyrene surfaces were treated with UV/ozone for ˜2.5 min through a mask of various geometries and then coated with various proteins: 20% human serum (v/v, batch 1, Sigma) for 1 hr at room temperature, 20% human serum (v/v, batch 2, Sigma) for 1 hr at room temperature, human vitronectin (Invitrogen; 1-3 μg/mL in DMEM) at 37° C. for 1 hr, or human vitronectin (Invitrogen; 1-3 mg/mL in DMEM) at 37° C. for 1 hr. These surfaces were then washed with cell culture medium before cell seeding.

Sorted hES BG01-Oct4-GFP cell line was plated as described above. Secondary fibroblasts derived from C1 cells were plated and cultured in 20% FBS in DMEM media as described previously.^{60,62}

Several different patterned cell cultures were generated through a combination of UV treatment and protein coating, e.g., with serum batch 1 or low vitronectin concentration or with serum 2 or high vitronectin concentration. Circular islands of adhesive surfaces in a repulsive background for hES cells and hES cells during gene targeting, and circular islands of repulsive surfaces in an adhesive background for hES cells and fibroblasts were observed.

UV-emission of adsorbed proteins was assayed for protein coating. UV emission of surfaces after coating indicated adsorption in spot areas in the case of human serum batch 1 and low vitronectin concentrations (<1 mg/mL). Mask and protein coating can be custom designed for any 2D geometric pattern.

Extended hES cell culture and clonal growth on UV/ozone patterned polystyrene. Virgin polystyrene surfaces were treated with UV/ozone for ˜2.5 min through a mask of 300 μm diameter and inter-spot spacing of 400 μm. Then, the surfaces were coated with human vitronectin (Invitrogen; 1-3 μg/mL in DMEM) at 37° C. for 1 hr. Cells were plated as discussed above, except that two different medias were used as indicated. Extended hES cell culture and clonal growth on UV/ozone pattered polystyrene was observed. hES cells were single cell seeded on UV/ozone-patterned polystyrene dishes and then grown for 7 days in either mTESR1 (Stemcell Technologies), fully-defined media or Nutristem (Stemgent) media. Dishes were pre-incubated with media with 20% human serum. The hESC clonal efficiency was determined as 27±11%, and this 20-30% clonal growth efficiency is comparable to traditional substrates utilizing mEFs.

Pluripotency phenotype is maintained upon extended cell culture and clonal growth on UV/ozone patterned polystyrene. Virgin polystyrene surfaces were treated with UV/ozone for ˜2.5 min through a mask of various geometries and then coated with human vitronectin (Invitrogen; 100 μg/mL in DMEM) at 37° C. for 1 hr. Cells were plated and cultured as discussed above. After seven days, cells were fixed in 4% paraformaldehyde in PBS and immunostained according to standard protocols using the following primary antibodies: SSEA4 (mouse monoclonal, Developmental Studies Hybridoma Bank); hSOX2 (goat polyclonal, R&D Systems); anti-GFP antibody (rabbit polyclonal, Abcam); hNANOG (goat polyclonal R&D Systems); appropriate Molecular Probes ALEXAFLUOR® dye conjugated secondary antibodies (Invitrogen) were used. When necessary, cells were permeabilized with 1% Triton X-100 in PBS for 10 mins, and then stained. Dishes were pre-incubated with media with 100 μg/mL vitronectin.

hES cells were single cell seeded on UV/ozone-patterned polystyrene dishes and then grown for 7 days in mTESR1, fully-defined media. Immunostaining for four different pluripotency markers: Oct4 (GFP), SSEA4, Nanog, and Sox2, indicated that the embryonic stem cell phenotype was robustly maintained.

Extended hiPS cell culture and clonal growth on UV/ozone patterned polystyrene. Ultra low attachment surfaces were treated with UV/ozone for ˜2.5 min through a mask of various geometries and then coated with human vitronectin (Invitrogen; 100 μg/mL in DMEM) at 37° C. for 1 hr. hiPS C1 cells were derived through lentiviral infection of Oct4, Sox2, and Klf4 and cultured in hES cell media on mEFs as described previously.^{60,62} At the time of this study, this line had been cultured for 5-15 passages on mEFs. This line was cultured in 10 μM Rho Kinase (ROCK) inhibitor (Calbiochem; Y-27632) for 24 hr in standard mEF conditions prior to sorting. Cells were harvested enzymatically with collagenase type IV (Invitrogen; 1 mg/ml), and then with 0.05% trypsin/ethylenediaminetetraacetic acid (EDTA) solution (Invitrogen) for 5 minutes at 37° C. Next, cells were labeled with immunostained using SSEA4 (mouse monoclonal, Developmental Studies Hybridoma Bank; 1:10 supernatant dilution in mTeSR1 media for 10-15 min at 4° C.) and Molecular Probes AlexaFluor 647 dye-conjugated secondary antibodies (Invitrogen; 1:50 for 10 min at 4° C.). Cells were collected in media with ROCK inhibitor and sorted on a FACSAria Flow Cytometer (Becton Dickinson, San Jose, Calif.). Cells were subsequently plated on various surfaces mTeSR1 media (Stemcell Technologies) supplemented with ROCK inhibitor for the first 24 hr. Culturing occurred in mTeSR1 media (Stemcell Technologies). After seven days of culture, the cells were fixed with 4% formaldehyde and stained using an Alkaline Phosphatase substrate kit I (Vector Labs) according to the manufacturer's procedure. Dishes were pre-incubated with media with 20% human serum.

C1 human induced pluripotent stem (hiPS) cells were single cell seeded on UV/ozone-patterned polystyrene dishes and then grown for 7 days in mTESR1, fully-defined media. Two different patterns were used: 300 μm spot diameter/200 μm spacing between spots and 300 μm spot diameter/400 μm spacing between spots. It was shown that pluripotency phenotype was maintained upon extended cell culture as the pluripotency marker, alkaline phosphatase (AP) was highly expressed.

Integrin-blocking cell behavior on UV/ozone-patterned polystyrene are similar to hit polymers. Cells were plated as discussed above. For the first 24 hrs, cells were blocked with anti-Integrin αᵥβ₅, clone P1F6, azide free antibody (19 μg/mL, Millipore, MAB1961Z) and/or anti-Integrin β₁ supernatant (1 mg/mL, Developmental Studies Hybridoma Bank, P5D2) in mTESR1 media. Cells were then fixed and counted manually. hES cells were single cell seeded on UV-patterned polystyrene dishes and then grown in the presence of various blocking antibodies for 24 hrs in mTESR1, fully-defined media. Dishes were pre-incubated with media with 20% human serum.

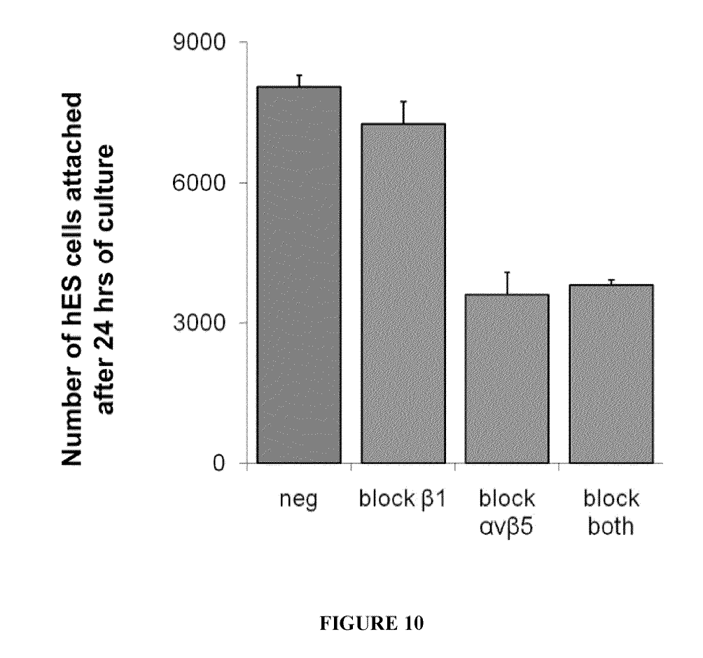

It was shown that cell adhesion is blocked only by the αᵥβ₅ integrin (vitronectin receptor) blocking antibody and not the β₁ blocking antibody (FIG. 10).

Somatic cell reprogramming on UV/ozone-patterned polystyrene dishes. Skin biopsies were obtained from an X-linked adrenoleukodystrophy adult patient with an ABCD1 exon 1 gene mutation, and fibroblasts outgrew from these biopsies in culture. Fibroblasts were then infected with lentiviral vectors as previously described.^{61,63,64} This procedure used multi-cistronic lentiviral vectors based on a combination of an IRES element and 2A peptide sequences to express multiple genes simultaneously from a single lentiviral vector⁶⁴ (termed “STEMCCA”). In this vector, two cistrons consist of Oct4 and Sox2 coding sequences fused to Klf4 and cMyc, respectively, through the use of intervening sequences encoding self-cleaving 2A peptides. Infected cells were then transferred to UV/ozone-patterned fibroblasts and grown in standard hES media. Surfaces were treated with UV/ozone for ˜2.5 min through a mask of various geometries and then coated with 20% human serum at 37° C. for 1 hr. Cells changed morphologies and colony-like structures after 2 weeks were manually picked and expanded under standard mEF culture conditions. Dishes were pre-incubated with media with 20% human serum.

It demonstrated that fibroblasts established from patient skin punch biopsy were reprogrammed to hiPSCs on patterned polystyrene (10 cm dish, 300 μm spot diameter/200 or 400 μm spacing) for 4 weeks. Here, skin biopsy from disease patient (adrenoleukodystrophy) was infected with reprogramming factors, and in day 1, it showed fibroblasts expressing reprogramming factors; in week 2, it showed fibroblasts expressing reprogramming factors, providing morphology changes; in week 3, it showed fibroblasts expression reprogramming factors, providing colony formation. The isolated clone was moved to mEFs in week 4 and disease-specific human induced pluripotent stem cell line was established.

Gene targeting of hES cells on UV/ozone-patterned polystyrene dishes. Zinc finger nucleases (ZFNs) against the human AAVS1 loci were designed using an archive of prevalidated two-finger modules exactly as described in published work.⁶³ The ZFNs were designed and tested at Sangamo BioSciences for the purpose of disruption of their intended target loci by transient transfection. BG01 hES cells were cultured in rho kinase (ROCK) inhibitor (Calbiochem; Y-27632) 24 h before electroporation.

Cells were harvested using 0.25% trypsin/EDTA solution (Invitrogen) and 1×10⁷ cells resuspended in PBS were electroporated with 40 μg of donor plasmids and 5 μg of each ZFN-encoding plasmid (Gene Pulser Xcell System, Bio-Rad; 250 V, 500 nF, 0.4-cm cuvettes). Donor plasmids consisted of a CAAGS promoter driving expression of GFP. Cells were subsequently plated on UV/ozone patterned polystyrene dishes in mTESR1 medium supplemented with ROCK inhibitor for the first 24 h. Individual colonies were picked and expanded after puromycin selection (0.5 μg/ml) 10-14 d after electroporation. Dishes were pre-incubated with media with 20% human serum.

Transgenic hES cells were generated by plating electroporated hES cells on patterned polystyrene (6 cm dish, 300 μm spot diameter/200 or 400 μm spacing) in day 1, and using zinc finger nuclease (ZFN)-mediated homologous recombination and drug selection culture media for 14 days. Rare transgenic cells grew upon drug selection during the 14 day culture.

Overview of human pluripotent cell directed differentiation on UV/ozone-patterned polystyrene dishes. hES cells were differentiated by plating hES cells on patterned polystyrene (10 cm dish, 300 μm spot diameter/400 or 200 μm spacing) and using appropriate culture medium to direct differentiation. It showed that ES or iPS cells were plated on patterned polystyrene and differentiated into ectodermal lineage to neural progenitors in neural differentiation media, differentiated into endodermal lineage to hepatocytes in hepatic differentiation media, or differentiated into mesodermal lineage to myeloid progenitor in hematopoietic differentiation media.

Ectodermal differentiation of hES cells on UV/ozone-patterned polystyrene dishes. For directed neural differentiation, hES cells were dissociated with Accutase (Invitrogen) for 15 minutes into a single cell suspension. MEFs were excluded by plating for one hour on gelatin at 37 C. The remaining pluripotent cells were plated on UV/ozone patterned surfaces in mTESR1 (Stemcell Technologies) at 3.5×10⁴ cells per cm². The cells were allowed to reach confluence in mTESR1 for 2-7 days, and shifted to KSR medium containing 10 μM SB431542 (Stemgent), and 500 ng/mL of Noggin (Stemgent). After 7 days of daily medium change, some cells were stained for Pax6 (covance rabbit anti-Pax6, 1:200) and Nestin (Chemicon, 1:200), followed by appropriate alexa-conjugated secondary antibodies (Invitrogen, 1:500). The large majority of cells expressed both markers of a neural progenitor fate. The remaining cells were adapted progressively to N2 medium, and finally passaged on day 10 to N2 medium containing 20 ng/mL EGF and 20 ng/mL βFGF to maintain the neural progenitor population. Dishes were pre-incubated with media with 20% human serum.

It was demonstrated that neural progenitor cells were generated by plating hES cells (e.g., BG01 hES cells and H9 hES cells) on patterned polystyrene (10 cm dish, 300 μm spot diameter/400 or 200 μm spacing) and using neural differentiation culture medium for 18 days. A human neural progenitor cell line was established.

Endodermal differentiation of hES cells on UV/ozone-patterned polystyrene dishes. For directed hepatic differentiation, hES cells were plated on UV/ozone-patterned polystyrene at 2.5×10⁴ cells per cm2 and cultivated under low oxygen conditions (4% O₂; 5% CO₂). Using standard protocols,⁶⁵ cells were passaged with Accutase (day 0) and differentiated in through the following steps: day 1-5, specify endoderm [20% O₂; 5% CO₂, RPMI/B27 media (Invitrogen) with Activin A (100 ng/ml)]; day 6-10, specify hepatic lineage [4% O₂; 5% CO₂, RPMI/B27 media (Invitrogen) with BMP4 (20 ng/ml; Peprotech) and FGF2 (10 ng/ml; Invitrogen)]; day 11-15, expand immature hepatocytes [4% O₂; 5% CO₂, RPMI/B27 media with hepatocyte growth factor (20 ng/ml; Peprotech)]; and, day 16-20: mature hepatocytes differentiation [20% O₂; 5% CO₂, Hepatocyte Culture media (Lonza) with Oncostatin-M (20 ng/ml; R&D Systems) and SingleQuots (without EGF)]. Dishes were pre-incubated with media with 20% human serum.

It was demonstrated that hepatocytes were generated by plating hES cells (e.g., H9 hES cells and BG01 hES cells) on patterned polystyrene (6 cm dish, 300 μm spot diameter/200 μm spacing) and using hepatocyte differentiation culture medium for 20 days.

Mesodermal differentiation of hES cells on UV/ozone-patterned polystyrene dishes. hES cells were plated on UV/ozone-patterned polystyrene dishes and cultivated in one of two media as indicated for seven days. The resulting cells can be used to generate more mature hematopoietic colonies by transferring them to Methocult GF⁺ media (StemCell Technologies) consisting of 1% methylcellulose, 30% FBS, 1% BSA, 50 ng/ml stem cell factor, 20 ng/ml granulocyte-macrophage colony-stimulating factor, 20 ng/ml IL-3, 20 ng/ml IL-6, 20 ng/ml granulocyte colony-stimulating factor, and 3 units/ml erythropoietin. Dishes were pre-incubated with media with 20% fetal bovine serum.

It was demonstrated that hematopoietic cells were generated by plating hES cells (H9 hES cells and BG01 hES cells) on patterned polystyrene (6 cm dish, 300 μm spot diameter/200 μm spacing) and using one of two different hematopoietic differentiation culture media for 14 days.

### REFERENCES

1. Amit, M. et al. Clonally derived human embryonic stem cell lines maintain pluripotency and proliferative potential for prolonged periods of culture. Dev Biol 227, 271-278 (2000).
2. Thomson, J. A. et al. Embryonic stem cell lines derived from human blastocysts. Science 282, 1145-1147 (1998).
3. Takahashi, K. et al. Induction of pluripotent stem cells from adult human fibroblasts by defined factors. Cell 131, 861-872 (2007).
4. Yu, J. et al. Induced pluripotent stem cell lines derived from human somatic cells. Science 318, 1917-1920 (2007).
5. Laflamme, M. A. et al. Cardiomyocytes derived from human embryonic stem cells in pro-survival factors enhance function of infarcted rat hearts. Nat Biotechnol 25, 1015-1024 (2007).
6. Lee, G. et al. Isolation and directed differentiation of neural crest stem cells derived from human embryonic stem cells. Nat Biotechnol 25, 1468-1475 (2007).
7. Hockemeyer, D. et al. Efficient targeting of expressed and silent genes in human ESCs and iPSCs using zinc-finger nucleases. Nature Biotechnology 27, 851-857 (2009).
8. Hanna, J. et al. Treatment of sickle cell anemia mouse model with iPS cells generated from autologous skin. Science 318, 1920-1923 (2007).
9. Kuehn, M. R., Bradley, A., Robertson, E. J. & Evans, M. J. A potential animal model for Lesch-Nyhan syndrome through introduction of HPRT mutations into mice. Nature 326, 295-298 (1987).
10. Zijlstra, M., Li, E., Sajjadi, F., Subramani, S. & Jaenisch, R. Germ-line transmission of a disrupted beta 2-microglobulin gene produced by homologous recombination in embryonic stem cells. Nature 342, 435-438 (1989).
11. Reeves, R. H. et al. A mouse model for Down syndrome exhibits learning and behaviour deficits. Nature Genetics 11, 177-184 (1995).
12. Gearhart, J. New potential for human embryonic stem cells. Science 282, 1061-1062 (1998).
13. Daley, G. Q. & Scadden, D. T. Prospects for stem cell-based therapy. Cell 132, 544-548 (2008).
14. Urbach, A., Schuldiner, M. & Benvenisty, N. Modeling for Lesch-Nyhan disease by gene targeting in human embryonic stem cells. Stem Cells 22, 635-641 (2004).
15. Ebert, A. D. et al. Induced pluripotent stem cells from a spinal muscular atrophy patient. Nature 457, 277-280 (2009).
16. Saha, K. & Jaenisch, R. Technical challenges in using human induced pluripotent stem cells to model disease. Cell Stem Cell 5, 584-595 (2009).
17. Zou, J. et al. Gene targeting of a disease-related gene in human induced pluripotent stem and embryonic stem cells. Cell Stem Cell 5, 97-110 (2009).
18. Zwaka, T. P. & Thomson, J. A. Homologous recombination in human embryonic stem cells. Nature Biotechnology 21, 319-321 (2003).
19. Martin, M. J., Muotri, A., Gage, F. & Varki, A. Human embryonic stem cells express an immunogenic nonhuman sialic acid. Nat Med 11, 228-232 (2005).
20. Xu, C. et al. Feeder-free growth of undifferentiated human embryonic stem cells. Nat Biotechnol 19, 971-974 (2001).
21. Richards, M., Fong, C. Y., Chan, W. K., Wong, P. C. & Bongso, A. Human feeders support prolonged undifferentiated growth of human inner cell masses and embryonic stem cells. Nat Biotechnol 20, 933-936 (2002).
22. Stojkovic, P. et al. Human-serum matrix supports undifferentiated growth of human embryonic stem cells. Stem Cells 23, 895-902 (2005).
23. Braam, S. R. et al. Recombinant vitronectin is a functionally defined substrate that supports human embryonic stem cell self-renewal via alphavbeta5 integrin. Stem Cells 26, 2257-2265 (2008).
24. Li, Y., Powell, S., Brunette, E., Lebkowski, J. & Mandalam, R. Expansion of human embryonic stem cells in defined serum-free medium devoid of animal-derived products. Biotechnol Bioeng 91, 688-698 (2005).
25. Amit, M., Shariki, C., Margulets, V. & Itskovitz-Eldor, J. Feeder layer- and serum-free culture of human embryonic stem cells. Biol Reprod 70, 837-845 (2004).
26. Yao, S. et al. Long-term self-renewal and directed differentiation of human embryonic stem cells in chemically defined conditions. Proc Natl Acad Sci USA 103, 6907-6912 (2006).
27. Ludwig, T. E. et al. Feeder-independent culture of human embryonic stem cells. Nat Methods 3, 637-646 (2006).
28. Ludwig, T. E. et al. Derivation of human embryonic stem cells in defined conditions. Nature Biotechnology 24, 185-187 (2006).
29. Li, Y. J., Chung, E. H., Rodriguez, R. T., Firpo, M. T. & Healy, K. E. Hydrogels as artificial matrices for human embryonic stem cell self-renewal. Journal of biomedical materials research Part A 79, 1-5 (2006).
30. Gerecht, S. et al. Hyaluronic acid hydrogel for controlled self-renewal and differentiation of human embryonic stem cells. Proc Natl Acad Sci USA 104, 11298-11303 (2007).
31. Watanabe, K. et al. A ROCK inhibitor permits survival of dissociated human embryonic stem cells. Nat Biotechnol 25, 681-686 (2007).
32. Hockemeyer, D. et al. Efficient targeting of expressed and silent genes in human ESCs and iPSCs using zinc-finger nucleases. Nat Biotechnol 27, 851-857 (2009).
33. Anderson, D. G., Levenberg, S. & Langer, R. Nanoliter-scale synthesis of arrayed biomaterials and application to human embryonic stem cells. Nat Biotechnol 22, 863-866 (2004).
34. Anderson, D. G., Putnam, D., Lavik, E. B., Mahmood, T. A. & Langer, R. Biomaterial microarrays: rapid, microscale screening of polymer-cell interaction. Biomaterials 26, 4892-4897 (2005).
35. Mei, Y. et al. Mapping the Interactions among Biomaterials, Adsorbed Proteins, and Human Embryonic Stem Cells. Advanced Materials 21, 2781-+(2009).
36. Green, J. J. et al. Nanoparticles for Gene Transfer to Human Embryonic Stem Cell Colonies. Nano Lett 8, 3126-3130 (2008).
37. Koenig, A. L., Gambillara, V. & Grainger, D. W. Correlating fibronectin adsorption with endothelial cell adhesion and signaling on polymer substrates. Journal of Biomedical Materials Research Part A 64A, 20-37 (2003).
38. Tamada, Y. & Ikada, Y. Effect of preadsorbed proteins on cell-adhesion to polymer surfaces. J Colloid Interf Sci 155, 334-339 (1993).
39. Underwood, P. A., Steele, J. G. & Dalton, B. A. Effects of polystyrene surface chemistry on the biological activity of solid phase fibronectin and vitronectin, analysed with monoclonal antibodies. Journal of Cell Science 104 (Pt 3), 793-803 (1993).
40. van Wachem, P. B. et al. The influence of protein adsorption on interactions of cultured human endothelial cells with polymers. J Biomed Mater Res 21, 701-718 (1987).
41. Keselowsky, B. G., Collard, D. M. & Garcia, A. J. Integrin binding specificity regulates biomaterial surface chemistry effects on cell differentiation. Proc Natl Acad Sci USA 102, 5953-5957 (2005).
42. Luther, E., Kamentsky, L., Henriksen, M. & Holden, E. Next-generation laser scanning cytometry. Cytometry, 4th Edition: New Developments 75, 185-218 (2004).
43. Urquhart, A. J. et al. High throughput surface characterisation of a combinatorial material library. Advanced Materials 19, 2486-+(2007).
44. Lipski, A. M. et al. Nanoscale engineering of biomaterial surfaces. Advanced Materials 19, 553-+(2007).
45. Zaari, N., Rajagopalan, P., Kim, S. K., Engler, A. J. & Wong, J. Y. Photopolymerization in microfluidic gradient generators: Microscale control of substrate compliance to manipulate cell response. Advanced Materials 16, 2133-+(2004).
46. Engler, A. J., Sen, S., Sweeney, H. L. & Discher, D. E. Matrix elasticity directs stem cell lineage specification. Cell 126, 677-689 (2006).
47. Saha, K. et al. Substrate modulus directs neural stem cell behavior. Biophys J 95, 4426-4438 (2008).
48. Tamada, Y. & Ikada, Y. Effect of Preadsorbed Proteins on Cell-Adhesion to Polymer Surfaces. Journal of Colloid and Interface Science 155, 334-339 (1993).
49. Thompson, M. T., Berg, M. C., Tobias, I. S., Rubner, M. F. & Van Vliet, K. J. Tuning compliance of nanoscale polyelectrolyte multilayers to modulate cell adhesion. Biomaterials 26, 6836-6845 (2005).
50. Delcorte, A. et al. ToF-SIMS study of alternate polyelectrolyte thin films: Chemical surface characterization and molecular secondary ions sampling depth. Surface Science 366, 149-165 (1996).
51. Vickerman, J. C. ToF-SIMS: surface analysis by mass spectrometry. (IM Publications, Charlton; 2001).
52. Urquhart, A. J. et al. TOF-SIMS analysis of a 576 micropatterned copolymer array to reveal surface moieties that control wettability. Analytical Chemistry 80, 135-142 (2008).
53. Meng, Y. et al. Characterization of integrin engagement during defined human embryonic stem cell culture. The FASEB Journal, 1-10 (2009).
54. Rowland, T. J. et al. Roles of Integrins in Human Induced Pluripotent Stem Cell Growth on Matrigel and Vitronectin. Stem Cells and Development (2009).
55. Hayman, E. G., Pierschbacher, M. D., Ohgren, Y. & Ruoslahti, E. Serum spreading factor (vitronectin) is present at the cell surface and in tissues. Proc Natl Acad Sci USA 80, 4003-4007 (1983).
56. Neuss, S. et al. Assessment of stem cell/biomaterial combinations for stem cell-based tissue engineering. Biomaterials 29, 302-313 (2008).
57. Constantinides, G., Kalcioglu, Z. I., McFarland, M., Smith, J. F. & Van Vliet, K. J. Probing mechanical properties of fully hydrated gels and biological tissues. Journal of biomechanics 41, 3285-3289 (2008).
58. Oliver, W. C. & Pharr, G. M. An improved technique for determining hardness and elastic modulus using load and displacement sensing indentation experiments. Journal of Materials Research 7, 1565 (1992).
59. Cheng, Y.-T. & Cheng, C.-M. Scaling, dimensional analysis, and indentation measurements. Materials Science and Engineering: R: Reports 44, 91-149 (2004).
60. Hockemeyer, D. et al. A drug-inducible system for direct reprogramming of human somatic cells to pluripotency. Cell Stem Cell 3, 346-353 (2008).
61. Hockemeyer, D., F. Soldner, et al. (2009). “Efficient targeting of expressed and silent genes in human ESCs and iPSCs using zinc-finger nucleases.” Nature Biotechnology 27(9): 851-7.
62. Hockemeyer, D., F. Soldner, et al. (2008). “A drug-inducible system for direct reprogramming of human somatic cells to pluripotency.” Cell Stem Cell 3(3): 346-53.
63. Soldner, F., D. Hockemeyer, et al. (2009). “Parkinson's disease patient-derived induced pluripotent stem cells free of viral reprogramming factors.” Cell 136(5): 964-77.
64. Sommer, C. A., M. Stadtfeld, et al. (2009). “Induced pluripotent stem cell generation using a single lentiviral stem cell cassette.” Stem Cells 27(3): 543-9.
65. Sullivan, G., D. Hay, et al. (2009). “Generation of functional human hepatic endoderm from human induced pluripotent stem cells.” Hepatology 50(12):1-7.

What is claimed is:

1. A device comprising a substrate adapted for culturing stem cells, and characterized by a secondary ion mass spectrometry (SIMS) ion signature correlated with desired stem cell culturing or differentiation efficiency.

2. The device according to claim 1, wherein the substrate comprises a polymer.

3. The device according to claim 2, wherein the substrate comprises an array of polymer domains distributed on a support.

4. The device according to claim 3, wherein the polymer is characterized by a secondary ion mass spectrometry (SIMS) ion signature comprising at least one of three most intense ion peaks selected from a hydrocarbon ion having no tertiary carbon atoms, a cyclic hydrocarbon ion, or an oxygen-containing ion derived from an ester.

5. The device according to claim 4, wherein the polymer comprises an acrylate-based polymer or copolymer having a SIMS ion signature comprising at least one of three most intense ion peaks selected from a C₁₋₄ hydrocarbon ion having no tertiary carbon atoms, a cyclic hydrocarbon ion, an oxygen-containing ion derived from an ester, O⁻, and OH⁻.

6. The device according to claim 5, wherein the SIMS ion signature comprises at least one of three most intense ion peaks selected from O⁻, C₂H⁻, OH⁻, CHO₂⁻, C₂H₃⁻, C₃H₅⁺, C₄H⁻, C₁₀H₁₁O⁻, CH⁻, C₃H₃⁻, C₃H₇⁺, C₂H₅O⁺, and C₂H₃O⁺.

7. The device according to claim 5, wherein the SIMS ion signature comprising a base peak and at least one of two subsequent ions according to peak intensity selected from O⁻, C₂H⁻, OH⁻, CHO₂⁻, C₂H₃⁻, C₃H₅⁺, C₄H⁻, C₁₀H₁₁O⁻, CH⁻, C₃H₃⁻, C₃H₇⁺, C₂H₅O⁺, and C₂H₃O⁺.

8. The device according to claim 5, wherein the SIMS ion signature comprising a base peak selected from O⁻, C₂H⁻, OH⁻, CHO₂⁻, C₂H₃⁻, C₃H₅⁺, C₄H⁻, C₁₀H₁₁O⁻, CH⁻, C₃H₃⁻, C₃H₇⁺, C₂H₅O⁺, and C₂H₃O⁺.

9. The device according to claim 5, wherein the SIMS ion signature comprising a base peak selected from O⁻, C₂H⁻, OH⁻, CHO₂⁻, C₂H₃⁻, and C₃H₅⁺.

10. The device according to claim 5, wherein the SIMS ion signature comprises the three most intense ion peaks selected from an ion other than CN⁻, C₂H₇O⁺, C₄H₉⁺, C₂H₆N⁺, C₃H₃O₂⁻, C₃H₈N⁺, C₅H₉⁺, C₅H₁₁⁺, CNO⁻, and C₃H₇O⁺.

11. The device according to claim 5, wherein the SIMS ion signature comprises the base peak selected from an ion other than CN⁻, C₂H₇O⁺, C₄H₉⁺, C₂H₆N⁺, C₃H₃O₂⁻, C₃H₈N⁺, C₅H₉⁺, C₅H₁₁⁺, CNO⁻, and C₃H₇O⁺.

12. The device according to claim 5, wherein the polymer domains have a water contact angle (WCA) from 45° to 90° C.

13. The device according to claim 5, wherein the acrylate-based polymer substrate comprises an array of at least 10 polymer domains distributed on a support, each domain having a major axis from 1 μm to 1000 μm.

14. The device according to claim 5, wherein the acrylate-based polymer substrate comprises an array of polymer domains, the array comprises a repeating microenvironment array adapted for maintenance or differentiation of human pluripotent stem cells, each microenvironment comprising the peripheral aspect of each polymer domain.

15. The device according to claim 5, wherein the polymer surface is coated by a protein component selected from serum, fibronectin, laminin, vitronectin, collagen, and any combination thereof.

16. The device according to claim 4, wherein the polymer comprises a styrene-based polymer having a SIMS ion signature comprising at least one of three most intense ion peaks selected from a C₂₋₆ hydrocarbon ion having no tertiary carbon atoms, a cyclic hydrocarbon ion, or an oxygen-containing ion derived from an ester.

17. The device according to claim 16, wherein the SIMS ion signature comprises at least one of three most intense ion peaks characterized by a carbon-to-hydrogen atomic ratio of less than 1.

18. The device according to claim 16, wherein the SIMS ion signature comprises at least two of three most intense ion peaks characterized by a carbon-to-hydrogen atomic ratio of less than 1.

19. The device according to claim 16, wherein the SIMS ion signature comprises at least one of three most intense ion peaks selected from C₂H₄O⁺, C₆H₉O⁺, C₃H₃O⁺, C₂H₃⁺, C₆H₁₁⁺, C₂H₅⁺, C₂H₃O⁺, C₅H₇O⁺, and C₃H₅⁺.

20. The device according to claim 16, wherein the SIMS ion signature comprising a base peak and at least one of the two subsequent ions according to peak intensity selected from C₂H₄O⁺, C₆H₉O⁺, C₃H₃O⁺, C₂H₃⁺, C₂F⁻, C₆H₁₁⁺, C₂H₅⁺, C₂H₃O⁺, C₅H₇O⁺, and C₃H₅⁺.

21. The device according to claim 16, wherein the SIMS ion signature comprising a base peak selected from C₂H₄O⁺, C₆H₉O⁺, C₃H₃O⁺, C₂H₃⁺, C₂F⁻, C₆H₁₁⁺, C₂H₅⁺, C₂H₃O⁺, C₅H₇O⁺, and C₃H₅⁺.

22. The device according to claim 16, wherein the SIMS ion signature comprises three most intense ion peaks selected from an ion other than C₇H₇⁺, CHO₂⁻, C₉H₉⁺, O⁻, C₇H₅O⁺, C₉H₇⁺, C₆H₅⁺, C₂H⁻, C₈H₇⁺, and C₇H₇O⁺.

23. The device according to claim 16, wherein the SIMS ion signature comprises the base peak selected from an ion other than C₇H₇⁺, CHO₂⁻, C₉H₉⁺, O⁻, C₇H₅O⁺, C₉H₇⁺, C₆H₅⁺, C₂H⁻, C₈H₇⁺, and C₇H₇O⁺.

24. The device according to claim 16, wherein the SIMS ion signature comprises the base peak selected from an ion other than C₇H₇⁺, CHO₂⁻, C₉H₉⁺, O⁻, C₇H₅O⁺, and C₉H₇⁺.

25. The device according to claim 16, wherein the polymer domains have a water contact angle (WCA) from 45° to 90° C.

26. The device according to claim 16, wherein the styrene-based polymer substrate comprises an array of at least 10 polymer domains distributed on a support, each domain having a major axis from 1 μm to 1000 μm.

27. The device according to claim 16, wherein the styrene-based polymer substrate comprises an array of polymer domains, the array comprises a repeating microenvironment array adapted for maintenance or propagation of human pluripotent stem cells, each microenvironment comprising the peripheral aspect of each polymer domain.

28. The device according to claim 16, wherein the polymer surface is coated by a protein component selected from serum, fibronectin, laminin, vitronectin, collagen, and any combination thereof.

29. The device according to claim 16, wherein the polymer is selected from a UV/ozone treated virgin bacterial grade polystyrene or UV/ozone treated ultralow attachment surface.

30. The device according to either claim 16, wherein the polymer comprises a UV/ozone-treated polystyrene.

31. A method of in vitro propagation or differentiation of stem cells, the method comprising culturing stem cells in a culture medium on a device comprising a substrate adapted for culturing stem cells, and characterized by a secondary ion mass spectrometry (SIMS) ion signature correlated with desired stem cell culturing or differentiation efficiency.

32. The method according to claim 31, wherein the device is adapted for clonal expansion of pluripotent stem cells, for somatic cell reprogramming to generate patient-specific hiPS cells, for gene targeting of hES cells, or for directed differentiation of hES cells into ectodermal, mesodermal, or endodermal lineages.

33. The method according to claim 31, wherein the substrate comprises a polymer.

34. The method according to claim 33, wherein the substrate comprises an array of polymer domains distributed on a support.

35. The method according to claim 34, wherein the polymer comprises a polymer characterized by a secondary ion mass spectrometry (SIMS) ion signature comprising at least one of three most intense ion peaks selected from a hydrocarbon ion having no tertiary carbon atoms, a cyclic hydrocarbon ion, or an oxygen-containing ion derived from an ester.

36. The method according to claim 35, wherein the polymer comprises an acrylate-based polymer having a SIMS ion signature comprising at least one of three most intense ion peaks selected from a C₁₋₄ hydrocarbon ion having no tertiary carbon atoms, a cyclic hydrocarbon ion, an oxygen-containing ion derived from an ester, O⁻, and OH⁻.

37. The method according to claim 36, wherein the substrate comprises an acrylate-based polymer having a SIMS ion signature comprising at least one of three most intense ion peaks selected from O⁻, C₂H⁻, OH⁻, CHO₂⁻, C₂H₃⁻, C₃H₅⁺, C₄H⁻, C₁₀H₁₁O⁻, CH⁻, C₃H₃⁻, C₃H₇⁺, C₂H₅O⁺, and C₂H₃O⁺.

38. The method according to claim 36, wherein the substrate comprises an acrylate-based polymer having a SIMS ion signature comprising three most intense ion peaks selected from an ion other than CN⁻, C₂H₇O⁺, C₄H₉⁺, C₂H₆N⁺, C₃H₃O₂⁻, C₃H₈N⁺, C₅H₉⁺, C₅H₁₁⁺, CNO⁻, and C₃H₇O⁺.

39. The method according to claim 35, wherein the polymer comprises a styrene-based polymer having a SIMS ion signature comprising at least one of three most intense ion peaks selected from a C₂₋₆ hydrocarbon ion having no tertiary carbon atoms, a cyclic hydrocarbon ion, or an oxygen-containing ion derived from an ester.

40. The method according to claim 39, wherein the polymer comprises a styrene-based polymer having a SIMS ion signature comprising at least one of three most intense ion peaks selected from C₂H₄O⁺, C₆H₉O⁺, C₃H₃O⁺, C₂H₃⁺, C₆H₁₁⁺, C₂H₅⁺, C₂H₃O⁺, C₅H₇O⁺, and C₃H₅⁺.

41. The method according to claim 39, wherein the polymer comprises a styrene-based polymer having a SIMS ion signature comprising three most intense ion peaks selected from an ion other than C₇H₇⁺, CHO₂⁻, C₉H₉⁺, O⁻, C₇H₅O⁺, C₉H₇⁺, C₆H₅⁺, C₂H⁻, C₈H₇⁺, and C₇H₇O⁺.

42. The method according to claim 31, wherein the substrate comprises a polymer surface that is coated by a protein component selected from serum, fibronectin, laminin, vitronectin, collagen, and any combination thereof.

43. The method according to claim 31, wherein the substrate comprises a polymer that is selected from a UV/ozone treated virgin bacterial grade polystyrene or UV/ozone treated ultralow attachment surface.

44. The method according to claim 43, wherein the polymer comprises a UV/ozone-treated polystyrene.
